(19) United States
(12) Patent Application Publication
(10) Pub. No.: US 2022/0184146 A1
(43) Pub. Date: Jun. 16, 2022

(54) OLEAGINOUS MICROBE SUPPLEMENTATION FOR IMPROVING BLACK SOLDIER FLY GROWTH AND DEVELOPMENT

(71) Applicants: Heather R. Jordan, Oktoc, MS (US); Jeffery K. Tomberlin, College Station, TX (US)

(72) Inventors: Heather R. Jordan, Oktoc, MS (US); Jeffery K. Tomberlin, College Station, TX (US)

(73) Assignees: MISSISSIPPI STATE UNIVERSITY, Mississippi State, MS (US); Texas A&M University, College Station, TX (US)

(21) Appl. No.: 17/551,271

(22) Filed: Dec. 15, 2021

Related U.S. Application Data
(60) Provisional application No. 63/126,338, filed on Dec. 16, 2020.

(51) Int. Cl. A61K 35/74; A61K 9/00; A61K 35/64; A23K 10/16; A23K 10/30; A23K 10/20

(52) U.S. Cl. CPC A61K 35/74; A61K 9/0056; A61K 35/64; A61K 2035/11; A23K 10/30; A23K 10/20; A23K 10/16

(57) ABSTRACT

The present invention provides a new method and composition to improve the growth, development, and/or nutritional value of a host organism, such as black soldier flies, utilizing oleaginous microbes, such as Arthrobacter and Rhodococcus species, to provide improved feed sources for animals and benefits to agriculture and energy.

### CROSS-REFERENCE TO RELATED APPLICATIONS

This application claims the benefit of U.S. provisional application No. 63/126,338 filed on Dec. 16, 2020, the entire disclosure of which is hereby incorporated by reference in its entirety as if fully set forth herein.

### FIELD OF THE INVENTION

This invention relates to the field of animal health and, more specifically, to a novel method, system, and composition to improve the growth, development, and/or nutritional value of a host organism, for example black soldier flies. The invention uses oleaginous microbes, such as bacterial supplements including Rhodococcus and Arthrobacter species, for example, to provide subsequently-improved feed source(s) for other animals. The process and system of the invention result in improved health and/or nutrition of the animals that consume such feed source(s).

### BACKGROUND OF THE INVENTION

The present invention relates to a novel methodology for supplementation of animal food utilizing oleaginous microbes to improve the growth, development, and nutritional value of black soldier flies, as one example of a host organism, to be used as a feedstock for other animals, thereby enhancing and improving the health and nutrition of those animals.

There are two problems currently afflicting the world that black soldier fly farming and associated research can help mitigate. The first, global demand for food produced for human consumption is predicted to increase by 100% over the span of the next 40 years. Despite efforts to keep up, it is predicted that agricultural production will not be sufficient to meet the demand, as demand is predicted to increase by 100-110% by 2050 and only a 60-70% increase is predicted in animal products in the same time period. Increased need for cattle and other animal proteins requires increased feed production with limited available land. The space and water requirements of both livestock and production of their feed accounts for nearly 70% of all the land used in agricultural production. Second, as both human and animal populations grow there will be an increase in waste production. Manure, food scrap waste, and agricultural waste all produce greenhouse gases and noxious odors as well as serve as potential incubators for pathogenic microbes. Therefore, safe and effective waste management solutions must be developed.

Entomophagy, the consumption of insects, has been practiced globally and throughout history, spanning nearly every culture. Insects are a great source of healthy fats, protein, and some trace elements. It can be considered more environmentally responsible to grow insects as a source of protein rather than rearing animal proteins because not only do insects require twelve times less feed and significantly less water to produce the same amount of protein as livestock, but they also produce significantly fewer greenhouse gas and ammonia emissions than any currently raised conventional livestock. Insects do not even have to totally replace animal proteins to be able to reduce the environmental impact of protein. Portions of the animals' diets can be substituted with insect proteins, and the animal waste can also be managed with insects which may then be used for feed. The low cost and space needs of insect rearing make it an ideal part of a solution for low-tech areas struggling with feed security. Also, insect rearing can be scaled up for higher production to address a global problem.

The black soldier fly (Hermetia illucens (L.)) is one of about 2000 species of insect that are used as food. The adult black soldier fly (BSF) is harmless, not a pest, is not a known vector of disease agents, and does not bite. Additionally, the adult black soldier fly does not take up any food and survives on reserves that it built up during its larval stage (BSFL). The larvae of the black soldier fly are known as voacious feeders that will consume and degrade most organic materials, with a material degradation percentage of from 55% up to 70%. They are able to degrade everything from fruit and vegetables to animal remains and manure. Black soldier flies have successfully been used to manage and degrade cow, chicken, and pig manure. These wastes can then be converted into insect biomass that is rich in both proteins and fats.

Substitution or partial replacement of traditional diets with BSFL has had positive results. Weaned pigs fed a diet consisting of 50% BSFL showed a 9% improvement in feed efficiency. Similarly, a study conducted on rainbow trout showed that replacing up to 40% of the fish's diet with BSFL showed no negative effects on both the fish's physiology and the quality of meat, but unfortunately lower levels of healthy polyunsaturated fatty acids were noted in the trout. BSFL have also been fed to poultry, usually because they are natural colonizers of poultry manure and have been used by farmers to help with waste management and prevent the manure from becoming a pollution issue. In many studies, BSFL were deemed a fit substitute for soybean or corn meal feed. When used to feed broiler quails, there was no difference in yield between quails whose diet had been partially replaced with BSFL and those who ate their usual diet, but the BSFL fed broiler quails did have improved amino acid levels that made the meat more nutritious and increased the saturated and monounsaturated fatty acids found in the meat. One more poultry study conducted with broiler chickens found that while feeding BSFL to the chickens did increase the levels of undesired fatty acids, defatting the BSFL decreased this effect.

BSFL are poised for mass production for proteins and oils as we know more about this species than any other species that offer the same potential. Numerous companies both in the United States and abroad are attempting to rear them for mass production as food, as feed, and as a waste management and conversion solution. However, the system has not been optimized for maximum production of proteins and lipids or for maximum waste degradation. The first step to their optimization is performing experiments on the benchtop, in order to determine variability and efficiency in methodology. It is important, however, to recognize the differences may be found when results at a small scale are compared to those obtained at the industrial scale. Reasons for this may include the sheer number of larvae in an industrial scale, as nutrient availability and access to food for each individual will differ from the small scale. In small scale studies, the larvae have less competition and easier access to food, as well as less surface area. On the industrial scale they must compete with thousands, not hundreds, of other larvae for resources. This in turn will influence waste conversion and feeding efficiency. Similarly, moisture content and the heat of the entire system will not be the same as on the benchtop because of the increased number of larvae seeking out food. Studies conducted on a small scale are important for initial results, determination of important variables, and fine-tuning methodology, but such studies will also need to be conducted on an industrial level before these methods can be considered for application to a “real world” or commercial setting.

BSFL have been shown to decrease the number of pathogens such as Salmonella enterica and Escherichia coli in its substrate and can become contaminated with the bacteria they encounter. Furthermore, studies have shown beneficial effects through bacterial supplementation. For instance, inoculating poultry manure used to raise BSFL with a bacteria, Bacillus subtilis, increased the growth of the larvae. Both of these studies show that these larvae can be influenced by probiotic additions. Probiotics are “viable microorganisms that, when ingested, have a beneficial effect”. In human intestinal health, probiotics are able to inhibit adherence of pathogens, compete for nutrients, and stimulate immunity. In insects, probiotics have been found to have beneficial effects. One study showed that Enterococcus kuehniella isolated from larval moth feces and orally administered to red flour beetle larvae increased infection survival rates of the beetle larvae due to the probiotic's antimicrobial activity. However, in another study bees fed sugar syrup supplemented with Lactobacillus rhamnosus (a commercially available probiotic) were more susceptible to disease and had a shorter lifespan. The latter study underscores the importance of probiotic selection in measuring health and functional outcomes.

Bacteria also provide nutrition in the form of triglycerides and lipids that are essential for insect growth and reproduction and can provide the energy needed during extended nonfeeding periods. This is particularly true during the larval stage where energy reserves are accumulated within the fat body to be utilized during metamorphosis. There is great diversity in the concentration of lipids present in bacterial species. For instance, oleaginous microbes have a high lipid content, which comprises about 20% or more of their biomass. Oleaginous microbes are excellent candidate organisms for the bioproces sing of chitinous waste (such as the exoskeletons of dead adult BSF), as many possess the enzymatic machinery to break down chitin and protein. Additionally, they can synthesize and accumulate triacylglycerides, similar in composition to vegetable oils, a primary material for biodiesel production. In a large scale nearly closed-loop system, rearing facilities could use adult flies allowed to emerge for breeding as a portion of the media used to grow the oleaginous microbes, eliminating waste output from the system and further supporting BSFL-rearing for protein as an environmentally conscious effort.

### SUMMARY OF THE INVENTION

The present invention provides a new method, system, and composition to improve the growth, development, and/or nutritional value of a host organism, for example black soldier flies. The invention uses oleaginous microbes, such as bacterial supplements including the Arthrobacter and Rhodococcus species, to provide an improved feed source(s) for other animals and other benefits to the fields of agriculture and energy, for example. The method, system, and composition(s) of the invention result in improved health and/or nutrition of the animals that consume such feed source(s).

In a first aspect, the invention relates to a method of feeding an animal including a step of feeding the animal a feed comprising oleaginous microbes selected from species of Arthrobacter, species of Rhodococcus and combinations thereof. In the method the oleaginous microbes may comprise from about 0.05 wt. % to about 20 wt. % of a total weight of the feed. The oleaginous microbes may include one or more bacteria selected from Arthrobacter AK19 and Rhodococcus rhodochrous 21198.

In a second aspect, the invention relates to a black soldier fly larva feed including:
a bulk feed suitable for a black soldier fly larva; and
a supplement comprising oleaginous microbes; and
   wherein the supplement comprises from about 0.05 wt. % to about 20 wt. % of a total weight of the black soldier fly larva feed.

The black soldier fly larva feed may include oleaginous microbes are selected from species of Arthrobacter, species of Rhodococcus and combinations thereof. The supplement may consist of oleaginous microbes. The oleaginous microbes may consist of species of Arthrobacter. Alternatively, the oleaginous microbes may consist of species of Rhodococcus. The black soldier fly larva feed may include one or more bacteria selected from Arthrobacter AK19 and Rhodococcus rhodochrous 21198.

The bulk feed may include one or more of alfalfa meal, corn meal, wheat bran, brewer's grain, manure, or food waste. The bulk feed may be about 30% alfalfa meal, about 20% corn meal, and about 50% wheat bran.

The supplement may comprise from about 0.1 wt. % to about 10 wt. % of the total weight of the black soldier fly larva feed.

The oleaginous microbes may be desiccated and subsequently rehydrated for use in the supplement.

In another aspect, the invention relates to a method of increasing the growth, development, or nutritional value of black soldier fly larva, includinging steps of:

mixing a bulk feed suitable for black soldier fly larvae with a supplement comprising oleaginous microbes to create a black soldier fly larva feed wherein the supplement is from about 0.05 wt. % to about 20 wt. % of a total weight of the black soldier fly larva feed, and

feeding the black soldier fly larva feed to black soldier fly larvae.

In the method, the oleaginous microbes may include species selected from species of Arthrobacter, species of Rhodococcus and combinations thereof. The supplement may consist of oleaginous microbes. The supplement may comprise from about 0.1 wt. % to about 10 wt. % of the total weight of the black soldier fly larva feed. The oleaginous microbes comprise one or more bacteria selected from Arthrobacter AK19 and Rhodococcus rhodochrous 21198.

The feeding may begin starting on day 11 after the black soldier fly larvae hatch.

In a further aspect, the invention relates to a method of increasing livestock production comprising a step of feeding livestock black soldier fly larva that were fed with the black soldier fly larva feed of claim 4.

With the foregoing and other objects, features, and advantages of the present invention that will become apparent hereinafter, the nature of the invention may be more clearly understood by reference to the following detailed description of the preferred embodiments of the invention and to the appended preferred embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing Black Soldier Fly Larval Growth over time when supplemented according to an embodiment of the invention.

FIG. 2 is a graph showing the ratio of waste:larval weight over time for Black Soldier Fly Larvae when supplemented according to an embodiment of the invention.

FIG. 3 is an overlay of separation profiles of larval proteins without and with supplementation according to an embodiment of the invention.

FIG. 4 shows the results of an expression analysis of major proteins present from the mass spectrometry analysis of proteins without and with supplementation according to an embodiment of the invention.

FIG. 5 is a graph showing the mean daily weights for larvae supplemented according to an embodiment of the invention compared to control larvae.

FIG. 6 is a graph showing the waste:larvae ratio for larvae supplemented according to an embodiment of the invention compared to control larvae.

FIG. 7A is a graph showing alpha diversity measures of number of observed species (Chaol) and abundance and evenness (Shannon) in supplemented larvae compared to control at small scale on day 7.

FIG. 7B is a graph showing alpha diversity measures of number of observed species (Chaol) and abundance and evenness (Shannon) in supplemented larvae compared to control at small scale on day 9.

FIG. 7C is a graph showing alpha diversity measures of number of observed species (Chaol) and abundance and evenness (Shannon) in supplemented larvae compared to control at small scale on day 10.

FIG. 8 is a graph showing the relative abundance of Family level microbial taxa from control or supplemented BSFL at small scale.

FIG. 9 is a graph showing the principal coordinate analysis (PCoA) of beta diversity between supplemented and control groups at days 7, 9 and 10 using Bray Curtis Distances. The percentage of total variation explained by each PCo axis is shown in the parentheses.

FIG. 10 is a graph showing the percent differences in predicted functions from microbial metagenomes of supplemented BSFL compared to control BSFL at small scale.

FIG. 11 is a graph showing the mean genome units of supplemented BSFL over time at small scale.

FIG. 12 is a graph showing mean weight over time of Bifidobacterium breve supplemented BSFL compared to control.

FIG. 13 is a graph showing the waste:larvae ratio of Bifidobacterium breve supplemented BSFL compared to control.

FIG. 14 is a graph showing the relative abundance of microbial families associated with Bifidobacterium supplemented BSFL compared to control on day 9.

FIG. 15 is a graph showing the percent difference in predicted functions from microbial metagenomes of Bifidobacterium supplemented BSFL compared to control on day 9.

FIG. 16 is a graph showing the mean larval weight (mg) of 100 Arthrobacter and Rhodococcus supplemented BSFL compared to control at industrial scale.

FIG. 17 is a graph showing the waste:larvae ratio of 100 Arthrobacter and Rhodococcus supplemented BSFL compared to control at industrial scale on day 10.

FIG. 18 is a graph showing the mean genome units of supplemented BSFL over time at industrial scale.

FIG. 19A is a graph showing alpha diversity measures of number of observed species (Chaol) and abundance and evenness (Shannon) in supplemented larvae compared to control at industrial scale on day 3.

FIG. 19B is a graph showing alpha diversity measures of number of observed species (Chaol) and abundance and evenness (Shannon) in supplemented larvae compared to control at industrial scale on day 6.

FIG. 19C is a graph showing alpha diversity measures of number of observed species (Chaol) and abundance and evenness (Shannon) in supplemented larvae compared to control at industrial scale on day 10.

FIG. 20 is a graph showing the relative abundance of Family level microbial taxa from control or supplemented BSFL at industrial scale.

FIG. 21 is a graph showing the principle coordinate analysis (PCoA) of beta diversity between supplemented and control groups at days 3, 6, and 10 using Bray Curtis Distances. The percentage of total variation explained by each PCo axis is shown in the parentheses.

FIG. 22 is a graph showing the percent differences in predicted functions from microbial metagenomes of Arthrobacter supplemented BSFL compared to control BSFL at industrial scale.

FIG. 23 is a graph showing the percent differences in predicted functions from microbial metagenomes of Rhodococcus supplemented BSFL compared to control BSFL at industrial scale

### DETAILED DESCRIPTION OF THE INVENTION

The present invention provides a new method, system, and composition to improve the growth, development, and/or nutritional value of a host organism, for example black soldier flies. The invention uses oleaginous microbes, such as bacterial supplements including the Arthrobacter and Rhodococcus species, for example, to provide an improved feed source(s) for other animals as well as other benefits to fields such as agriculture and energy. The method, system, and composition(s) of the invention may result in improved health and/or nutrition of the animals that consume such feed source(s).

The addition of fat-rich bacteria to the feeding substrate of BSFL increases body mass, development rate, feed-to-body mass conversion, and nutrient density of the BSFL. The present invention uses the effectiveness of bacterial supplementation to BSFL food to improve BSFL growth and waste conversion, and the recognizes the importance of scale and probiotic choice in feeding experiments.

Black soldier flies are poised for mass production of protein or oils, as they can be produced at the tonnage level daily, and more is known about their biology than any other insect species that could be utilized for this purpose. Black soldier flies can be mass-produced and can convert organic wastes to protein and fat and reduce dry matter by 50%. Black soldier fly prepupae can be used as feed for a variety of fish and could provide a 25 to 50% replacement of current feed used in the aquaculture industry. As fish meal and other protein sources for aquaculture feed become scarcer and more expensive, insect protein sources offer a much-needed alternative. Protein can also be separated from the chitinous cuticle of the black soldier fly and used as additional foodstuff, leading to greater replacement. Additionally, black soldier flies have recently been approved by the American Association of Feed Control Officials and Canadian Food Inspection Agency for use as feed for salmonid fish species (AAFCO, 2016) and by the Federal Drug Administration for poultry feed, underscoring the importance of black soldier fly utility. However, the system has not been optimized for maximum protein and fat yields, or for waste degradation.

Remediating waste is of paramount importance to concentrated animal feeding operations (CAFOs), such as dairies. Therefore, an economical method for remediation, while protecting natural resources is essential to the long-term health of CAFOs and the environment. The present invention shows that oleaginous microbe supplementation allows black soldier fly larvae to metabolize more of the food than control larvae fed the same diet without supplementation. These results show that oleaginous microbe supplementation allow black soldier fly larvae to digest higher concentrations of manure. Probiotics are live microorganisms that, when administered in adequate amounts, confer a health benefit on the host. All members of the Animalia kingdom, including humans, have helpful symbiotic microbiota which are extremely important for the proper functioning of the gastrointestinal tract, and now many such products are being commonly used as dietary supplements. Probiotics are recommended to be added not only to the human diet but also into the forage of different vertebrates as well as invertebrates.

Increased scrutiny of human or animal microbiota has led to the identification of novel probiotics, such as in this study, that are not members of the traditional genera (i.e., Lactobacillus or Bifidobacterium in other systems). The studies presented here showed that oleaginous microbes should be used as a probiotic species for the black soldier fly because of their positive effects on nutrition and development.

In recent years and in many countries, public concern about the safety of foods of animal origin has heightened due to problems that have arisen with contamination, outbreaks of food borne bacterial infections, and due to a growing concern about drug residues and microbial resistance to antibiotics. These problems have drawn attention to practices within the feed and livestock industries and have prompted health professionals and the feed industry to closely examine food quality and safety issues. Notably, black soldier fly larvae present no known harm to the environment or people, as they are a non-pest species and have been found to suppress pathogens in waste.

The present data shows that black soldier fly feed supplemented with R. rhodochrous may increase larval lipid and protein content, feeding efficiency and waste conversion. Oleaginous microbes may also be excellent candidate organisms for the bioprocessing of chitinous waste, as many possess the enzymatic machinery to break down chitin and proteins. For instance, the oleaginous yeast Cryptococcus curvatus can produce intracellular lipid using chitin as a carbon source and the total lipid content can reach 54% on a chitin rich medium. R. rhodochrous was found to use chitin as a carbon source in a present study (data not shown).

Another attractive feature is that oleaginous microbes can synthesize and accumulate triacylglycerides, similar in composition to vegetable oils, a primary material for biodiesel production. Altogether, food waste supplementation with oleaginous microbes provides added value to the system through increasing black soldier fly lipid and protein content, along with increasing feeding efficiency and waste conversion. Furthermore, the breakdown of chitinous byproducts into lipids and proteins may convert a low value resource to a high value resource for the farmer. Data from the present study shows the impact and driving mechanisms of food waste supplementation with oleaginous microbes on black soldier fly waste conversion and resulting nutritional composition.

The addition of oleaginous microbes such as those of the Arthrobacter and Rhodococcus species increase BSFL mass. Arthrobacter AK19 was chosen for additional studies at small scale because the bacterium possesses a high concentration of lipids, usually accumulating greater than 40% lipids in dry biomass.

Not to be bound by theory, Arthrobacter is a well-characterized microbe commonly found in soil and in decomposition environments and may be breaking down a grain-based diet, making it easier for the larvae to absorb feed associated nutrients. Arthrobacter also has commercial uses for the production of L-glutamate, and has been found to be nutritionally versatile, utilizing a variety of substrates. Arthrobacter can reduce a variety of aromatic compounds, herbicides and pesticides, hexavalent chromium and 4-chlorophenol in contaminated soil, increasing interest in their use in bioremediation. Most species of Arthrobacter are obligate aerobes, but all exhibit a pure respiratory, never fermentative metabolism, and some strains have been found to grow anaerobically, utilizing nitrate as their terminal electron acceptor.

Rhodococcus is a related genera to Arthrobacter and has similar biodegradation capabilities. Furthermore, Arthrobacter and Rhodococcus species can degrade lignocellulosic biomass for lipid biosynthesis.

As shown in Example 1, the inventors conducted a 10-day study comparing growth and developmental stage of 11-day old larvae fed a routine diet (Gainesville Diet) alone versus an5 autoclaved or non-autoclaved diet with Rhodococcus rhodochrous supplementation. Results showed that larval weight was significantly higher among those with bacterial supplementation (p=0.025 and 0.028) autoclaved and non-autoclaved food with bacterial supplementation, respectively). Data also showed that larvae with bacterial supplementation metabolized more food than larvae fed the Gainesville Diet alone. Furthermore, bacterial supplementation allowed faster larval development.

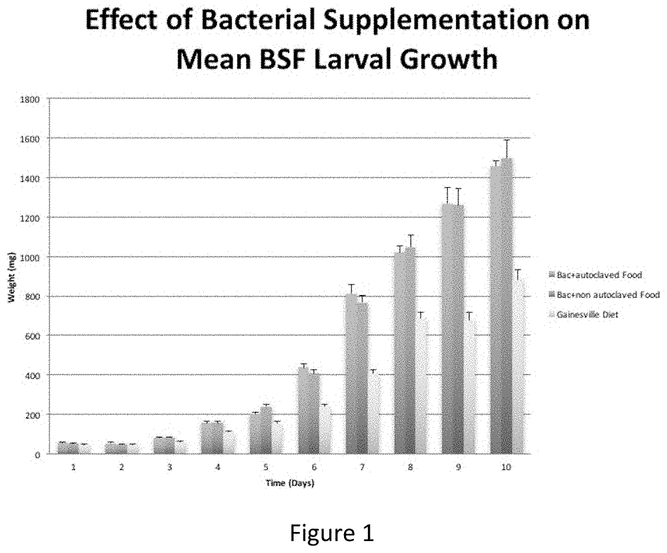

Supplementing the diet provided to black soldier fly larvae with R. rhodochrous significantly enhances development as well as protein and fat production. This supplementation is shown in FIG. 1 to result in a near doubling (1.7×) in larval weight. Also, larvae reached a mean weight of 120 mg per individual larvae, which is the target weight for commercial use in the R. rhodochrous supplemented treatments between days 8 and 9, whereas mean larval weights in the control treatment did not reach 120 mg per individual larvae during the duration of the study. These data show that bacterial supplementation allows for greater metabolization of the diet and allows for a lesser quantity of food needed to reach optimal weight. This is important when considering industrial scale production of larvae for commercial use.

Analysis of fatty acids also revealed that bacterial supplementation increased the concentration of short-chain saturated fatty acids, and reduced the amount of unsaturated fatty acids, increasing utility for the biodiesel industry. Moreover, the bacteria-supplemented diet also shows detectable levels of vaccenic and eicosatrienoic acids, which are unsaturated fatty acids implicated in potential health benefits. This shows that probiotics may be used to optimize black soldier fly industrialization for the production of protein for feed and fat for bioenergy. Doing so may allow for agriculture to more easily meet the needs of billions of people who still lack access to basic, modern energy services while simultaneously meeting food demands and participating in a global transition to clean, low-carbon energy systems.

Proteomic analysis of the black soldier flies with or without R. rhodochrous supplementation led to many significantly differentially expressed proteins, when compared to the control black soldier flies fed the Gainesville Diet alone. Interesting findings from this study showed that control black soldier fly larvae reared without R. rhodochrous supplementation expressed a larger number of proteins involved in energy production and storage, as well as muscle development and contraction. Additionally, those control larvae without R. rhodochrous supplementation had significantly greater expression of heat shock proteins. And, as previously mentioned, they also weighed less and metabolized less food than those supplemented with the bacteria. Not to be bound by theory, we hypothesize black soldier fly larvae fed the Gainesville diet alone were more active in searching for access to key nutrients than those provided the R. rhodochrous supplementation, which released these bound nutrients to the larvae. Furthermore, black soldier fly larvae fed with R. rhodochrous supplementation also significantly and differentially expressed histone proteins. These proteins are part of the nucleosome and are instrumental in giving structure to DNA. This increase is likely due to an increase in chromatin content and may be an indirect marker for cell proliferation (i.e., growth).

In addition to these data, calmodulin was found significantly expressed in black soldier fly larvae with R. rhodochrous supplementation. Calmodulin binds calcium and has been implicated in inflammation, metabolism, and smooth muscle contraction. Calcium is also required in many cases for initiation of DNA synthesis. The potential of calmodulin as a growth modulator has already been demonstrated in other systems. Since black soldier fly larvae are known to be high in calcium, the potential link with the inventor's discovery is that bacterial supplementation enhances calcium deposition in the exoskeleton

As shown in Examples 2-4, a small-scale study was conducted with a different oleaginous microbe used for supplementation. Additionally, another small-scale study was conducted using a different probiotic. Finally, an industrial scale experiment was conducted utilizing a supplement containing the same two oleaginous species as used in Examples 1 and 2, but at a larger scale. Results were similar to the small-scale experiments where bacterial supplementation allowed faster development and increased waste conversion. It was also determined that desiccated R. rhodochrous and Arthrobacter can be rehydrolized and fed to larvae described above to yield the same outcome.

As a result, black soldier fly larvae can be enhanced and improved for animal feed as a protein replacement. These data show that bacterial supplementation with the bacterium used, for example, can increase larval growth and speed up development, thus reducing the amount of food needed to reach higher weights and desired growth stages for protein and oil isolation.

The Arthrobacter supplemented larvae significantly increased in weight from day 3 onward to day 10 compared to the no supplementation control larvae. The treated larvae appeared to be tinted orange (Arthrobacter grows bright orange colonies) and just as active as the control larvae. Not to be bound by theory, the oleaginous nature of Arthrobacter AK19 and larval continuous feeding may have allowed the larvae to store energy and nutrients in their fat body. Ingesting such a “fat microbe” may aid in increasing BSFL fat stores. Additionally, Arthrobacter could be “pre-digesting” the food for the larvae, allowing an increase in nutrient availability.

Another potential explanation is that Arthrobacter and Rhodococcus are colonizing the gut of the larvae and, like human probiotics, assisting with the digestive process. The present data indicate that Arthrobacter may be colonizing the gut, as Arthrobacter were detected by qPCR throughout the course of both studies. However, Rhodococcus was only detected by qPCR on day 3 of the experiment, and was below detectable limits at the remaining timepoints, suggesting only transient Rhodococcus passage through the gut. Another possible explanation is that Arthrobacter and Rhodococcus changes the initial environmental conditions allowing other bacteria to proliferate, and may be maintained in the BSFL waste, particularly as the waste becomes more alkaline. Day 7 of the small-scale study yielded percent increases in all functional groups as compared to control. This is reflective of both the taxa present as well as the relative abundance of those taxa. At day 7, there was an increase in abundance of Actinomycetaceae, Alcaligenaceae, Brucellaceae, Corynebacteriaceae, Flavobacteriaceae, Neisseriaceae, Sphingobacteriaceae, and Staphylococcaceae.

The 16s sequencing data included families where Arthrobacter and Rhodococcus reside, but detected only a 184 Micrococcaceae (Family for Arthrobacter, data not shown) combined abundance from all treatment samples, and at all timepoints for the small-scale study. Micrococcaceae was also detected from sequencing industrial scale larval guts, and found a 635 combined abundance with larval gut treatments, with detected abundance at day 6 and day 10 of the study. Nocardiaceae (Family for Rhodococcus, data not shown) were also detected at every timepoint, with a combined abundance of 957, with decreased detection at later timepoints. Differences in sensitivity and specificity of the two methods, along with relative abundance associated with 16s sequencing, where increase of one taxon leads to the equivalent decrease of remaining taxa, likely account for these differences.

Amino acid metabolism is responsible for breaking down protein present in feed into amino acids or di- or tripeptides. Energy metabolism on the other hand is crucial for ATP production as well as purine and pyrimidine synthesis which is a substrate for nucleic acids. Furthermore, lipid metabolism aids in bacterial cell membranes. An increase in lipid metabolism in the Arthrobacter supplement group is likely due to the increase in Sphingobacteriaceae, which contain high concentrations of cellular lipids. However, there was a decrease in Bogoriellaceae, a relatively obscure bacterial family, and Enterococcaceae, which could be driving the percent decrease in lipid metabolism and bile secretion at day 7. Changes in the environmental substrate during larval feeding also likely led to changes in species composition and relative abundance. Species responses to stimuli including changes in water availability, pH, temperature, toxicity, and nutrient availability would be important in this system as these changes are observed throughout the course of larval feeding. Organisms present in high abundance in the Arthrobacter supplemented groups appeared to have broad systems for responding to these changes including those for bacterial motility and signaling, antimicrobial resistance and biosynthesis, and pollutant/contaminant degradation. An in-depth look at predicted genes within the functional groups showed enrichment for two component systems, and a higher abundance of bacterial motility and flagella proteins and bacterial secretion systems compared to the control, which play important roles in bacterial attachment, colonization, and chemotaxis. Many of the identified microbial families have been found to be involved in gut digestion in mammals and other animals, as well as degradation of organic aromatic compounds and other organic pollutants. Additionally, many of these are known to produce antimicrobial and other secondary compounds.

At day 10, control samples showed an increase in abundance of Corynebacteriaceae, and Staphylococcaceae, while Arthrobacter treated groups showed a large increase in abundance of Enterococcaceae compared to the control. Accordingly, most Arthrobacter supplemented BSFL microbiome functional groups showed large percent decreases in abundance compared to controls, with only bile secretion and nucleic acid repair/replication/general metabolism increased.

The change in predicted genes associated with bile acid biosynthesis and bile secretion was an interesting finding across all treatments and experiments. Many bacterial families identified include bacterial species associated with bile acid biosynthesis and digestion in mammals, where clear interactions between bile and gut bacteria have been identified. In mammals, many gut bacteria utilize bile and produce secondary bile acids. Some studies have shown that an increase in bile leads to an increase in gram positive members of Firmicutes, from which families such as Clostridiaceae, Bacilliaceae, Enterococcaceae and Staphylococcaceae reside. Bile acids have antimicrobial effects on gut microbes, while also aiding in host digestion through emulsifying fats and oils, to allow further nutritional processing. A role of bile and bile acids as hormones and signaling molecules in bacteria has also been identified that act on various physiological functions. And, a dynamic equilibrium has been found to exist between diet-gut microbiome-bile acid pool size and composition, and perturbations in this equilibrium can result in disease states. While insects do not produce bile, emulsifiers have been identified in invertebrates that aid in digestion. It is therefore possible that some of the microbial families identified in studies presented in this work are utilizing insect emulsifiers as carbon sources and may be aiding in insect digestion or lipid storage within the fat body. However, a literature search revealed very little data describing natural invertebrate lipid emulsifiers as a whole, and it is not known what enzymes, outside of general lipases, are utilized for lipid digestion and storage in BSFL.

Taken together, these small-scale results with Arthrobacter are promising and, along with previous small scale Rhodococcus data, point toward the potential for industrialization of this process. Larvae that reach harvestable sizes sooner save industrial BSF production companies money and increase their yield. If the larvae are being used for waste management, their organic material degradation ability can be increased with the aid of these probiotics, helping them to process more waste in a shorter amount of time, with the possibility of degradation of intractable materials through bacterial supplementation directly, or indirectly through a change in microbial populations toward those with these traits.

The effect that B. breve supplementation had on the larvae was unexpected. It was expected that addition of this probiotic would have a positive effect on the larvae in some way, just like this probiotic can aid digestion in humans. However, this was not the case. Supplemented larvae appeared discolored, slow, covered in a sticky exudate and overall unhealthy (data not shown). They stuck to each other, to the feeding substrate, and to the container. Healthy control larvae were tan-colored, active and moved through their feeding substrate without issue. The daily mean weight of supplemented larvae was lower than the control larvae, and their waste:larvae ratio was high. Bifidobacterium breve did not aid the larvae in converting their food to body mass. Additionally, Bifidobacterium treatments showed an increase in Clostridiaceae, and closer inspection revealed an increase in the Clostridium genera. And despite the increase in Promicromonosporaceae and Cellumonadaciae which contain species with high concentrations of cellulases and xylanases, digestion and frass excretion also appeared stalled. A literature search of studies in other animal models revealed that administration of high fat diets can lead to a decrease in growth among Bifidobacterium strains, and also a reduction in weight in obese individuals. Additionally, studies have shown that with these same high fat diets, a disproportionate increase in propionate and acetate producing species, including Clostridiales, Bacteroides, and Enterobacteriales can arise. Bifidobacterium has been suggested to be associated with changes in inflammatory markers associated with obesity. An additional mechanism hypothesized here includes fat malabsorption and excretion within the insect frass, though more in-depth studies should be conducted to validate this hypothesis. So, in this sense, administration of Bifidobacterium kept the BSFL “skinny”, as has been shown to occur in other animal and human studies. However, in the case of BSFL, this result is not conducive for overall insect health if the goal is to increase growth and waste conversion. However, supplementation with B. breve may be useful if the goal is to slow growth and development. Additionally, data from this work suggests a new mechanism for B. breve's role in decreasing obesity.

The industrial scale experiment included both Arthrobacter and Rhodococcus as treatments. The mean daily weights showed that Rhodococcus and Arthrobacter supplemented larvae were consistently higher than control larvae throughout the study. Control larvae initially had lower weights than both the Rhodococcus and Arthrobacter treatment, and only overtook them on the very last day. Not to be bound by theory, a potential explanation for the decrease in mean weights on the last day could be attributed to pupation. As the BSFL prepares for pupation it moves into the prepupal stage. In this stage larvae stop feeding and their integument begins to harden. Their digestive system empties, and they exhibit a crawl-off behavior as they seek out a safe place away from the feeding substrate where they can pupate. If the Arthrobacter and Rhodococcus supplementation was able to accelerate development, then it would undergo this process sooner. When we collected samples to weigh, they would have been in an advanced stage and likely weighing less. Additionally, there was no statistical differences in the waste:larvae ratios between the groups at industrial scale. This was not surprising since the waste:larvae ratio was only measured on day 10, where there were no differences in the larval weights for that timepoint. However, because the Arthrobacter supplemented group still showed a better conversion ratio even at that timepoint, it is believed that statistically significant differences in waste:larval ratios would have been found if measured at earlier timepoints when there was less variation between replicates.

Bacterial supplementation also yielded changes in microbiome species presence and relative abundance, similar to what was found at small scale, with the greatest differences found from day 6 samples. At this time point, treatment groups showed increases in Sphingobacteriaceae and Staphylococcaceae, as well as Pseudomodadaceae, which was absent from the control samples. Predicted genes involved in all functional groups were also present. Planococcaceae and Bacilliaceae were more abundant in controls at this timepoint, and were likely contributors to the percent decrease in treatments in predicted genes involved in energy metabolism and motility and signaling, particularly with those genes involved in sporulation found in Bacillus ssp. At day 10, observed species were the same for all groups, however, relative abundance differed, where control samples showed an increase in relative abundance in Enterococcaceae, Planococcaceae, Pseudomonadaceae, and Xanthamondadacea, which suggests these are the primary taxa contributing to the percent decrease in predicted gene functions in the treatment groups compared to controls.

Bacterial supplementation yielded somewhat comparable results at small and large scales, depending on the timepoints. For instance, on day 3, Arthrobacter supplemented BSFL weighed 21% versus 22% more than control BSFL, and on day 10, 11% versus 6.7% more than control BSFL at bench and industrial scale. And on day 6, Arthrobacter supplemented BSFL weighed 35% versus 29% more than control BSFL at benchtop versus industrial scale, respectively. Bacterial supplementation at industrial scale also yielded changes in gut microbiome species presence and relative abundance, with the greatest differences found from day 6 samples, whereas large differences in gut microbiome relative abundance were also observed at day 7 of the benchtop experiment. Predicted genes involved in all functional groups were also present, similar to benchtop scale. But, control and treatment larvae during both benchtop scale experiments had not reached peak weight and were still growing at the final timepoint. In contrast, treatment larvae at industrial scale had reached peak weight at day 6 of the experiment with an increased number of pupated larvae (data not shown), whereas control larvae at the industrial scale were also still growing. Differences in scale, numbers of larvae, amount of substrate, and differing inoculum for benchtop and industrial scale experiments likely account for this.

Changes in the environmental substrate during larval feeding and through supplementation likely led to changes in species composition and relative abundance. BSFL responses to stimuli including changes in water availability, pH, temperature, toxicity, and nutrient availability would be important in this system as these changes are observed throughout the course of larval feeding. Organisms present in high abundance in the bacterial supplemented groups appeared to have broad systems for responding to these changes including those for bacterial motility and signaling, antimicrobial resistance and biosynthesis, and pollutant/contaminant degradation. An in-depth look at predicted genes within the functional g0roups showed enrichment for two component systems, and higher abundance of bacterial motility and flagella proteins and bacterial secretion systems compared to control, which play important roles in bacterial attachment, colonization, and chemotaxis. Many of the identified microbial families have been found to be involved in gut digestion in mammals and other animals, as well as degradation of organic aromatic compounds and other organic pollutants. Additionally, many of these are known to produce antimicrobial and other secondary compounds.

Overall, bacterial supplementation is beneficial to BSFL larval growth and waste conversion, though care should be taken toward the appropriate bacterial supplementation. Bacterial supplementation yields comparable results at small and large scales, though there was a difference in some bacterial taxa identified among microbiomes. This may be due to differences in feed batches or larvae initial microbiomes.

Based on the results from the studies, the present invention is a black soldier fly larva feed. The larva feed includes a bulk feed source and a supplement. To increase larval growth the supplement includes oleaginous microbes. Preferably the oleaginous microbes are of the species Arthrobacter and Rhodococcus. Either a single species or a combination of species may be used for the bacterial supplement. Additionally, other probiotics such as Pseudomonas putida, could be used in conjunction with the oleaginous microbes to provide additional benefits to the BSFL or to the end consumer, such as livestock.

Preferably, the supplement is employed in an amount of about 0.05 wt. % to about 20 wt. % of the total weight of the larva feed. More preferably, the supplement comprises from about 0.1 wt. % to about 10 wt. %, and most preferably, from about 0.1 wt. % to about 1 wt. % of the total weight of the larva feed.

The bulk feed source may be any feed source that is known in the art to be used as feed for BSFL. Preferably, such feed source includes any combination of alfalfa meal, corn meal, wheat bran, brewer's grain, manure, or food waste. More preferably, the bulk feed is about 30% alfalfa meal, about 20% corn meal, and about 50% wheat bran (Gainesville Diet).

An embodiment of the invention also includes a method for increasing the growth, development, or nutritional value of black soldier fly larva by feeding the larva a diet of bulk feed supplemented with a supplement comprising oleaginous microbes. The larva is preferably fed the supplemented diet from 11 days after hatching until harvest or pupation.

To provide the downstream benefit to livestock farming, the larva that consumed the supplemented feed are then used as a substitute protein source to feed to livestock.

Other important data will include transcriptomic data. While useful for our study, PICRUSt is limited in that genes may not be transcribed or translated, limiting the impact of their annotated function. Nevertheless, changes in functional predictions in these datasets could be related to relative abundance differences across time and treatment, based on gene annotations for a given taxa, giving insight into microbially mediated mechanisms of BSFL feeding and waste conversion. Another interesting finding was the number of taxa with functional potential for pollutant/contaminant degradation. This was an exciting finding in that there is further potential of specific bacterial supplementation, particularly many of those enriched within our studies, and manipulation in the BSFL system to allow BSFL to degrade intractable materials and also have potential utility in bioremediation, while also increasing proteins and lipids of value.

### EXAMPLES

### Example 1

Bacteria culturing. Rhodococcus rhodochrous 21198 was grown on Luria nutrient agar and broth at pH 6.8 and 26° C. for three days, then collected by either scraping the plates or centrifuging the broth and collecting the pellet. All of the collected bacteria were washed in a saline solution to remove residual nutrient media.

Black Soldier Fly Rearing. Black soldier fly eggs were collected from a colony maintained in the Forensic Laboratory for Investigative Entomological Sciences (FLIES) Facility at Texas A&M University using methods described by (Sheppard et al., 2002). The eggs were collected in three layers of 2 (w) by 2 (h)×3 (1) cm corrugated cardboard blocks taped to the inside a 2 L plastic bucket 3 cm above approximately 500 g of the Gainesville diet (30% alfalfa meal, 20% corn meal, and 50% wheat bran) saturated with water (Hogsette, 1992). Cardboard was replaced daily. Cardboard containing eggs were placed in a ˜1 L deli cup and maintained in an incubator at approximately 70% RH, 27° C., and 12:12 L:D until hatched.

Experiment Design and Data Collection. Eleven day old larvae were sent to Mississippi State University where allotments of 300 larvae were placed into three separate replicate containers for each of three treatments that were fed daily 6.0 g: 1) Gainesville diet (control) at 70% saturation (with molecular grade water); 2) heat killed Gainesville diet with 10% R. rhodochrous supplementation (0.6 g), and 3) non-heat killed Gainesville diet with 10% R. rhodochrous supplementation (0.6 g). Each replicate had a perforated plastic wrap securing the top to prevent escape and placed in a climate-controlled room with 60% relative humidity, 28° C. and 12:12 L:D.

Weight of the total larvae in each replicate was taken in triplicate and recorded at the initiation of the experiment. Larvae were separated from food waste and weighed every 24 h and then returned to their respective cup with fresh food. Food waste was separated daily from each cup and was dried for three hours in a MyTemp Mini Digital Incubator (Benchmark Scientific) set at 50° C., then weighed as well. After 10 d, the experiment was concluded given larval weight had plateaued. Resulting larvae were sacrificed and analyzed as described below. Nutritional Assessment. Four larvae per replicate were randomly selected and pooled for each timepoint at the conclusion of the experiment and were sent to the University of Arizona for protein analysis, and to Microbial ID, Inc for fatty acid methyl ester (FAME) analysis. Protein analyses were conducted using a Dionex UltiMate 3000 (Thermo Scientific) high performance liquid chromatography system (HPLC) coupled with an LTQ Velos Pro (Thermo Scientific) tandem mass spectrometer. Raw mass spectra was analyzed using the X!tandem and OMSSA algorithms (Wright et al., 2016). Differential expression analysis of proteins between treatments was performed pairwise using peptide elution profiles as described previously and below (Wright et al., 2016).

Microbial ID conducted FAME analysis using the Sherlock Microbial Identification System (MIS). This system uses Sherlock Software version 6.3, with a Hewlett Packard HP6890 Gas Chromatograph, with GC ChemStation Rev. B. 04.03 for naming peaks, and associated methods, but was adapted for BSF (Buyer and Sasser, 2012). Briefly, Sherlock MIS uses fatty acids 9-20 carbons in length. The peaks are automatically named and quantitated by the system. The Sherlock MIS used an external calibration standard developed and manufactured by Microbial ID, Inc. The standard consists of a mixture of the straight chained saturated fatty acids from 9 to 20 carbons in length (9:0 to 20:0) and five hydroxy acids. All compounds were added quantitatively so the gas chromatographic performance could be evaluated at the time the calibration mixture was analyzed. The hydroxy compounds are especially sensitive to changes in pressure/temperature relationship and to contamination of the injection port liner. As a result, compounds functioned as quality control checks for the system. Retention time data obtained from injecting the calibration mixture were converted to Equivalent Chain Length (ECL) data for fatty acid naming. The Sherlock libraries consist of more than 100,000 analyses obtained from experts and collections. Analysis of an unknown sample resulted in an automatic comparison of the composition of the unknown to a database using a covariance matrix, principal component analysis and pattern recognition software. The covariance matrix considers the mole-for-mole relationship of the conversion of one fatty acid to another (e.g. 16:0 to 16:1 due to action of a desaturase), which might occur in relation to a temperature shift or age difference. The pattern recognition software uses calculations of cross terms (e.g. ratios between fatty acid amounts) in addition to the principal component base. The libraries are open ended and are only limited by MIDI's ability to obtain adequate numbers of samples to make the entries. Statistical Analysis. Data from life history experiments and FAME profiles were analyzed using IBM SPSS Statistics for Windows, version 24.0 (IBM Corp., Armonk, N.Y., USA). Data were first tested for normality then analyzed by Analysis of variance (ANOVA) to determine significant differences between and within treatments with a Bonferroni's post hoc test when significant effects were observed. Variables for analyses included: Treatment (feed substrate type), fatty acids, total residue, waste reduction, and larval weight. Significance was defined as p<0.05. Replicate data variability was represented by error bars on graphs as standard error of the mean.

Differential expression of proteins between groups was performed pairwise using peptide elution profiles as described previously in Wright, M. L., Pendarvis, K., Nanduri, B., Edelmann, M. J., Jenkins, H. N, Reddy, J. S., Wilson, J. G., Ding, X., Broadway, P. R., Ammari, M. G., Paul, O., Roberts, B. and Donaldson, J. R., 2016. The Effect of Oxygen on Bile Resistance in Listeria monocytogenes. J Proteomics Bioinform 9: 107-119. 10.4172/jpb.1000396. Briefly, precursor mass spectra were extracted from the raw data using the MSConvert GUI software from the ProteoWizard toolset. Peptide precursor m/z values were extracted from protein identifications using Perl. Elution profiles for peptide-spectrum matches were calculated by parsing each corresponding MS1 file and summing the ion current for that match's m/z value within a 0.25 Da tolerance, effectively integrating the elution profiles. Multiple peptide-spectrum matches with the same precursor m/z were only counted once, ensuring the same integral was not included multiple times. Intensities were summed for each protein on a per-replicate basis. Proteins not identified in a replicate were represented with the average noise level of the replicate's chromatogram for further calculations. Data were normalized using a mode-based technique, where the mode of the protein intensities for each replicate was calculated, representing the most commonly occurring protein intensity, and the intensity per replicate divided by the mode of the same replicate to ensure normalization was not affected by the minimum and maximum intensities. A permutation analysis was performed for each protein by evaluating the difference in means of the replicates of both conditions. From this permutation, a p-value was calculated to indicate the significance of the difference in means. Two additional permutations were performed for each protein, comparing both conditions to their own baselines. These baseline permutations provided a mechanism to further reduce false positives introduced by differences in chromatogram ion current as electron multiplier performance deceases. Proteins were considered to be differentially expressed if the difference in means between conditions resulted in a P<0.05 and the difference in means between one of the conditions and its baseline was P<0.05.

### Results

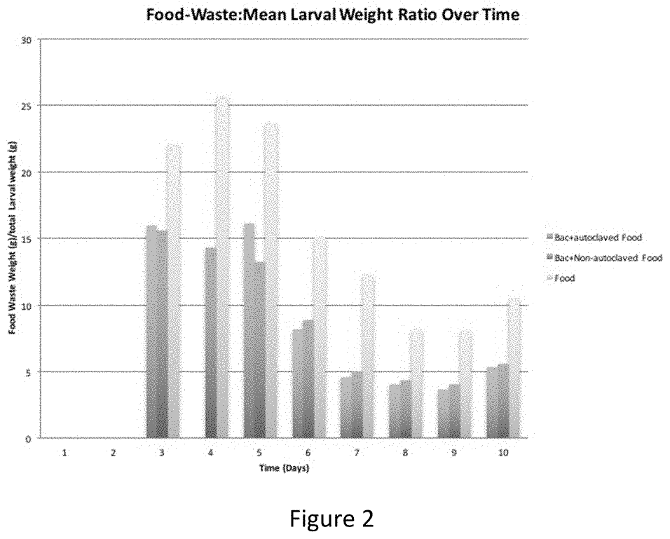

Life-History Traits and Waste Conversion. There was no statistical difference between the weights of the two R. rhodochrous treatments during the entire 10-day experiment. However, larval weight from the R. rhodochrous+nonautoclaved diet treatment group was significantly higher than the control beginning at day 3 (F_{2,23}=6.49; p=0.004 Bonferroni post hoc) as shown in FIG. 1, and larval weights from both R. rhodochrous treatment groups were significantly higher than control weights from day 4 through day 10 (F_{2,23}=14.63 (day 4), 18.04 (day 5), 36.31 (day 6), 52.08 (day 7), 31.16 (day 8), 14.82 (day 9), 25.53 (day 10), p<0.001 for both treatments from days 4 to 10). Additionally, weight of food-waste was divided by the total larval weight in each container to determine conversion rate. The R. rhodochrous treatment groups metabolized significantly more food and had a significantly higher waste conversion than control larvae fed Gainesville Diet alone beginning on day 5 and day 6 through day 10 for R. rhodochrous+nonautoclaved diet treatment group and R. rhodochrous+autoclaved diet treatment groups, respectively (F_{2,23}=31.68 (day 5), 78.51 (day 6), 128.18 (day 7), 50.00 (day 8), 66.78 (day 9), 15.47 (day 10), p<0.001 for all) as shown in FIG. 2.

Fatty Acid Composition. The detailed percent fatty acid composition of BSF with or without R. rhodochrous supplementation is shown in Table 1.

TABLE 1
Comparison of mean composition of representative fatty acids
from larvae fed bacteria supplemented diet compared to with diet alone.

| | | BSF Larvae + | |
|---|---|---|---|
| | | Probiotic | BSF Larvae + |
| Fatty Acids | | Supplemented Diet | Diet Alone |
| Lauric | C12:0 | 55.2 | 51.4 |
| Myristic | C14:0 | 0.34 | 0.25 |
| Palmitic | C16:0 | 8.9 | 9.8 |
| Palmitoleic | C16:1n-7 | 2.4 | 2.8 |
| Stearic | C18:0 | 1.2 | 1.4 |
| Vaccenic | C18:1n-7 | 0.88 | 0.00 |
| Oleic | C18:1n-9 | 8.7 | 11.4 |
| Linoleic | C18:2n-6 | 7.6 | 10.2 |
| Eicosatrienoic | C20:3n-3 | 0.35 | 0.08 |
| Saturated fatty acids* | | 79.2 | 75.0 |
| (SFA) | | | |
| Monounsaturated | | 13.1 | 14.7 |
| fatty acids* (MUFA) | | | |
| Polyunsaturated | | 7.9 | 10.3 |
| fatty acids* (PUFA) | | | |

*Values are total composition and include all detected fatty acids.

No statistical differences (F_{1,334}=0.001, p=0.998) were found between fatty acid composition of R. rhodochrous with autoclaved or non-autoclaved food; therefore, the two treatments were combined, and the mean taken. Fatty acid composition of larvae fed with R. rhodochrous supplementation showed higher saturated fatty acids (79.2%) than those fed Gainesville diet alone (75%). However, larvae fed with R. rhodochrous supplementation had lower monounsaturated fatty acids (MUFAs) (13.1% versus 14.7%) and polyunsaturated fatty acids (PUFAs) than control larvae (7.9% versus 10.3%). R. rhodochrous supplemented larvae also showed increased % concentration of short-chain saturated fatty acids when compared to control larvae (65.64% versus 62.85%). Larvae fed R. rhodochrous supplemented diet also showed detectable levels of vaccenic (0.88% versus 0.0%) and eicosatrienoic acids (0.35% versus 0.08%), which were absent, or present only in a very small amount, respectively, in control larvae fed diet alone.

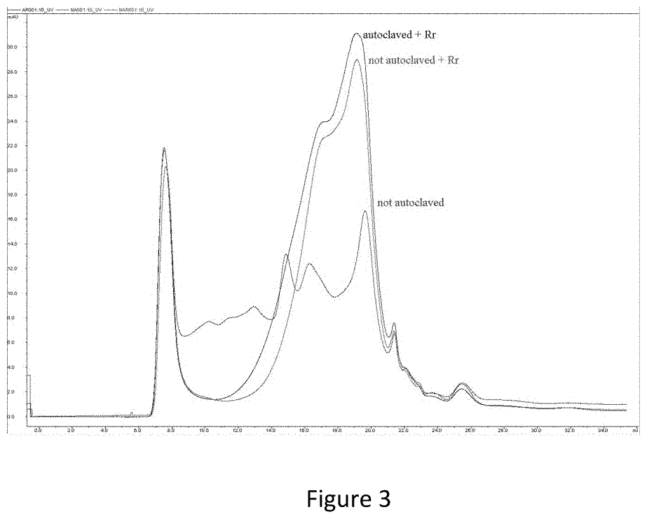

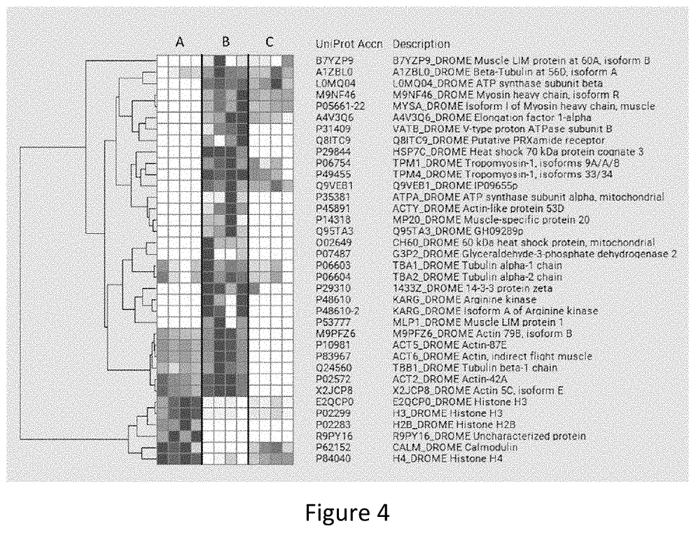

Protein Expression Profiling. The separation profiles of larval proteins with and without R. rhodochrous supplementation are shown in FIG. 3. The overlay of the sample peaks with 280 nm absorption demonstrates differences in protein profiles associated with R. rhodochrous supplementation versus larval proteins fed Gainesville Diet alone. Results showed overlap in the initial peaks (6.0-10.0 fracions) from all treatments, though control larval proteins and proteins from R. rhodochrous+autoclaved proteins had peaks at higher absorbance as compared to the R. rhodochrous+nonautoclaved diet (22.0 versus 20.0 absorbance). The second major peaks were identified for fractions 12.0 through 24.0 and showed differences between R. rhodochrous supplemented larval proteins compared to control larval proteins. R. rhodochrous+autoclaved diet had a peak at 31.0 absorbance and R. rhodochrous+nonautoclaved diet showed a peak at 29.0 absorbance. However, control larval proteins from these fractions showed three smaller peaks at 12.0, 13.0 and 18.0 absorbance. Protein extracts from the larvae were analyzed using mass spectrometry in order to determine relative amounts of major proteins present and normalized significantly different proteins from expression analysis are shown in FIG. 4.

Thirty-six proteins were significantly differentially expressed between R. rhodochrous treatments and controls. Overall, 18 of the 36 proteins, particularly those involving actin, myosin, and tubulin that are involved in muscle development, contraction, and organization. Twelve of the 36 differentially expressed proteins are known to be involved in energy production and storage, protein synthesis, and DNA packaging. Two of the 36 proteins were identified as heat shock proteins, and one was identified as calmodulin, a calcium binding protein.

Results show many differentially expressed proteins that are found in black soldier fly larvae fed Gainesville Diet alone (FIG. 4, Panel B), that are absent from those fed with R. rhodochrous supplementation (FIG. 4, panel A and C). Many of them are involved in muscle development, contraction, and organization. Notably, heat shock 70 kDA protein cognate 3 and 60 kDA heat shock protein (mitochondrial) were expressed in control BSF larvae fed diet alone, but not expressed in those fed with R. rhodochrous supplementation. ATP synthase subunit alpha (mitochondrial), and glyceraldehyde-3 phosphate dehydrogenase 2 were also significantly expressed in the control diet only cohort alone. Additionally, arginine kinase and isoform A of arginine kinase, both energy storage compounds, were found in control larvae fed diet alone. Proteins significantly expressed in BSF larvae fed with R. rhodochrous supplementation, while very low or absent in the control, diet alone cohort included histone proteins, H3, H2B (with supplemented, autoclaved diet only), and H4. Additionally, calmodulin was significantly expressed in the R. rhodochrous supplement cohorts, but not expressed or expressed below detection limits in the control, diet alone cohort.

### Examples 2-4

### 1.1 Fly Colony

Black soldier fly eggs were collected from a colony at the Forensic Laboratory for Investigative Entomological Sciences (FLIES) facility at Texas A&M University. Eggs were collected in three layers of 2×3 cm corrugated cardboard blocks placed above approximately 500 g of spent grain diet saturated with water. The cardboard was replaced daily, and cardboard containing eggs was placed in a one-liter deli cup and held in an incubator at 70% relative humidity, 27° C. and 12:12 L:D until the eggs have hatched. The larvae were shipped to Mississippi State University Department of Biological Sciences when the larvae were eleven days old for each of the experiments conducted at Mississippi State University.

### 1.2 Bacterial Growth and Collection

Both Rhodococcus rhodochrous 21198 and Arthrobacter AK19 were grown on Luria nutrient agar and broth at pH 6.8 and 26° C. for three days, then collected by either scraping the plates or centrifuging the broth and collecting the pellet. All of the collected bacteria were washed in a saline solution to remove residual nutrient media. Bifidobacterium breve was grown anaerobically at 37° C. on plates and collected by scraping plates.

### 1.3 DNA Extraction

DNA from the subsets of larvae and waste were isolated using a modified protocol of that discussed Williamson, H. R. et al. Mycobacterium ulcerans fails to infect through skin abrasions in a guinea pig infection model: implications for transmission. PLoS Negl Trop Dis 8, e2770 (2014)., quantified by a Qubit 2.0, and purified using a Qiagen DNA clean-up kit. Genomic DNA was extracted from all replicates and the extracts were subsequently pooled. The DNA was amplified with V4 primers and suggested protocols by Thompson, L. R. et al. A communal catalogue reveals Earth's multiscale microbial diversity. Nature 551, 457-463 (2017) and visualized by gel electrophoresis. Verified amplifying DNA was sent to Michigan State University Sequencing Facility for paired-end 16S metagenome sequencing.

### 1.4 DNA Sequencing

Microbial DNA samples were sequenced using Illumina MiSeq of 2×250 bp paired-end reads following 16S library construction, both performed by the Michigan State University Genomics Core Facility. The V4 hypervariable region of the 16S rRNA gene was amplified using dual indexed Illumina compatible primers 515f/806r as described by Kozich, J. J., Westcott, S. L., Baxter, N. T., Highlander, S. K. & Schloss, P. D. Development of a dual-index sequencing strategy and curation pipeline for analyzing amplicon sequence data on the MiSeq Illumina sequencing platform. Appl. Environ. Microbiol. 79, 5512-5120 (2013). PCR products were normalized using Invitrogen SequalPrep DNA Normalization plates and the products recovered from the plates pooled. This pool was cleaned up with AMPureXP magnetic SPRI beads. The pool was QC'd and quantified using a combination of Qubit dsDNA HS, Advanced Analytical Fragment Analyzer High Sensitivity NGS DNA and Kapa Illumina Library Quantification qPCR assays. Sequencing of the pooled amplicons was on an Illumina MiSeq v2 standard flow cell using a 500 cycle v2 reagent cartridge. Custom Sequencing and index primers were added to appropriate wells of the reagent cartridge as described in Kozich, J. J., Westcott, S. L., Baxter, N. T., Highlander, S. K. & Schloss, P. D. Development of a dual-index sequencing strategy and curation pipeline for analyzing amplicon sequence data on the MiSeq Illumina sequencing platform. Appl. Environ. Microbiol. 79, 5512-5120 (2013). Base calling was done by Illumina Real Time Analysis (RTA) v1.18.54 and output of RTA was demultiplexed and converted to FastQ format with Illumina Bcl2fastq v2.19.1.

Raw FastQ files barcoded Illumina 16S rRNA paired-end reads were assembled, quality-filtered, demultiplexed, and analyzed in QIIME version 1.8.0. Reads were discarded if they have a quality score <Q20, contained ambiguous base calls or barcode/primer errors, and/or were reads with <75% (of total read length) consecutive high-quality base calls. Chimeric reads were removed using the default settings in QIIME. After quality control, the remaining sequences were binned into OTUs at a 97% sequence similarity cutoff using UCLUST. Assembled sequence reads were classified into Operational Taxonomic Units (OTUs) on the basis of sequence similarity. The highest-quality sequences from each OTU cluster were taxonomically assigned using the RDP classifier and identified using BLAST against reference sequences from the most current Greengenes 97% reference dataset (http://greengenes.secondgenome.com). Representative sequences of all OTUs were aligned to the Greengenes reference alignment using PyNAST, and low abundance OTU's (<0.0005% of reads in the total dataset) were removed. Samples were rarefied to achieve equal coverage per sample and those samples with fewer sequences were used in subsequent analyses.

Sequence Archiving: Sequences were archived within the NCBI Sequence Read Archive (https://submit.ncbi.nlm.nih.gov/subs/sra/SUB135510/overview) under Accession Number: PRJNA663337.
Quantitative PCR for Detection of Arthrobacter and Rhodococcus Over Time. Primers were designed for targeting the respective 16s regions of Rhodococcus rhodochrous and Arthrobacter AK-19. Primers and Taqman probe targeting R. rhodochrous 21198 16s included forward primer: 5′ACGACGTCAAGTCATCATGC; reverse primer: 5′ GTATCGCAGCCCTCTGTACC; probe (VIC fluorophore): VICTATGTCCAGGGCTTCACACAMGBNFQ. Primers and Taqman probe targeting Arthrobacter AK-19 16s included forward primer: 5′ GTGGGTACGGGCAGACAGA; reverse primer: 5′ CTACGCATTTCACCGCTACA; probe (FAM fluorophore): 6FAMGTGCAGTAGGGGAGACTGGAMGBNFQ. Ten-fold dilutions were created for standards using known concentrations of R. rhodochrous 21198 and Arthrobacter AK-19. Standards were analyzed in triplicate, samples were analyzed in duplicate, and qPCR reactions were multiplexed. Conditions for qPCR included 3 μL of template, 1 μL each of forward and reverse primers (2.5 μM), 2.5 μL each of probe (0.125 nM), and 12.5 μL Environmental MasterMix (ThermoFisher). Cycling conditions included an initial melting temperature of 95° C. for 3 minutes, following by 40 cycles of 95° C. for 1 minutes, 55° C. for 30 seconds, and 72° C. for 45 seconds.

### 1.5 Analyses of Microbial Diversity

Bacterial diversity was assessed through the Chaol estimator and the Shannon index, calculating both indexes after subsampling with QIIME and data against the Greengenes Database, to avoid sequencing effort bias. Relative abundance was also assessed and plotted at family level using the R vegan and phyloseq statistical packages. Family level abundance less than 2% were not shown. Principal Coordinates Analysis (PCoA) and Bray-Curtis dissimilarity index were used from the R statistical package to study community composition, assessing the statistical significance of the differences in sample groupings through Bray-Curtis distance matrices and Adonis testing. Statistical tests used in the study were two-sided, and a p value of 0.05 or less was reported as statistically significant.

### 1.6 Determination of Functional Capacity Using Phylogenetic Investigation of Communities by Reconstruction of Unobserved States (PICRUSt)

In order to predict genes from the metagenome, closed-reference operational taxonomic units (OTUs) were obtained from the filtered reads using QIIME version 1.8.0. The biom-formatted OTUs table was then loaded to PICRUSt on the online Galaxy version in the Langille Lab (v1.1.1), alongside the Greengenes database (last updated June 2017). PICRUSt software estimates functional potential from the community metagenome using copy normalized 16S rRNA sequencing data whose gene contents are contributing to KEGG (Kyoto Encyclopedia of Genes and Genomes) identified pathways. Functionally annotated genes that were identified were compressed into 12 general gene families. Comparisons were made between differences in annotated gene abundance from control and treatment groups to determine the percent change of treatment as compared to control groups. Only those with gene abundance at or above 25% change were considered for analysis.

### Example 2

### Arthrobacter AK19 Supplementation: Small Scale

Eleven-day old BSFL were sent to MSU from Dr. Tomberlin's lab at Texas A&M Un iversity. Larvae were divided into sets of 300 larvae, with each set placed into control or treatment containers in triplicate. A perforated plastic wrap was secured on the top of the containers to prevent escape. Larvae in control containers were fed daily with 18.0 g Gainesville diet (a standard plant-based diet composed of 30% alfalfa meal, 20% corn meal, and 50% wheat bran with water), while treatment containers were fed 16.65 g Gainesville diet supplemented with 1.35 g (7.5% of diet) of Arthrobacter AK19. The control diets received additional water in place of a supplement to make up for the moisture difference. Initial weights of the larvae were recorded by randomly selecting 25 larvae from the containers and weighing them, as well as initial weight of the diets. Every 24 hours the larvae were separated from their feeding substrate. The feed and waste in the container were weighed, as well as the larvae in sets of 25. Containers were kept in a controlled and constant environment at room temperature. After 10 days, the experiment was stopped and the larvae and waste were immediately weighed. Waste from each replicate was collected, weighed, and dried at constant temperature (55° C.) for 24H then weighed again. Remaining larvae and waste from immediate collection were frozen in −20 C until further analysis.

### Results

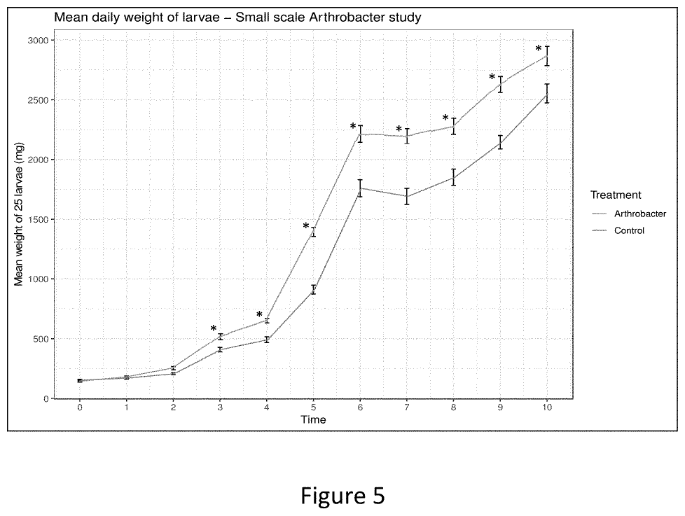

The mean daily weights for the small scale Arthrobacter AK19 supplementation study shows the mean daily weight with standard errors bars as shown in FIG. 5. The mean daily mass of supplemented larvae was greater than that of the control-diet larvae, particularly at early timepoints. On the third day, treatment groups were 94% larger than control in mass and had increased 107% from day 2 to day 3, whereas control larvae only increased mass by 28% from day 2 to day 3. A similar but steadily diminishing trend was seen in later timepoints: On day 5, treatment larvae were 58% larger than control larvae and increased their mass by 113% from day 4. Despite control larvae having an 85% increase in mass from the day 4 timepoint, their overall mass was still less than treatment larvae. There was a significant difference in mean daily weight between treatment and control larvae at day 3 (p=0.007), day 4 (p=0.0003), day 5 (p=0.005), day 6 (p=0.001), day 7 (p=0.0006), day 8 (p=0.007) day 9 (p=0.002) and day 10 (p=0.015).

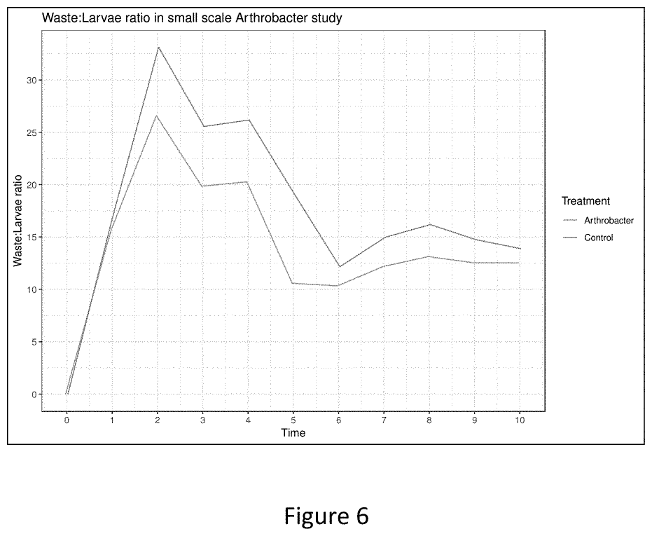

The waste:larvae ratio was calculated for timepoints 2-10 and shows the ability of the larvae to convert their feeding substrate (Gainesville Diet) into body mass. These results are shown in FIG. 6. An overall lower waste:larvae ratio was observed in Arthrobacter supplemented groups, revealing that the bacterial supplemented larvae had an increased capacity to break down and ingest the feeding substrate and convert it to biomass. The waste:larvae ratio increased during the first few days, and peaked at day 2. As the larvae continued feeding on their food and received daily feedings, the ratio decreased until the larvae neared the prepupal stage from T6-T10.

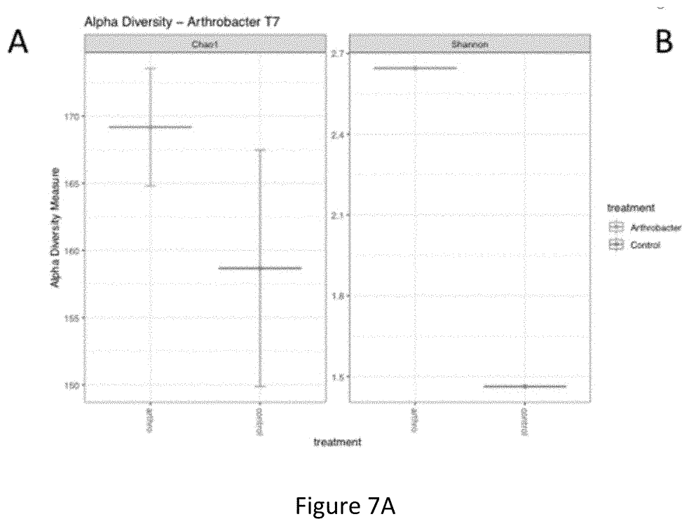

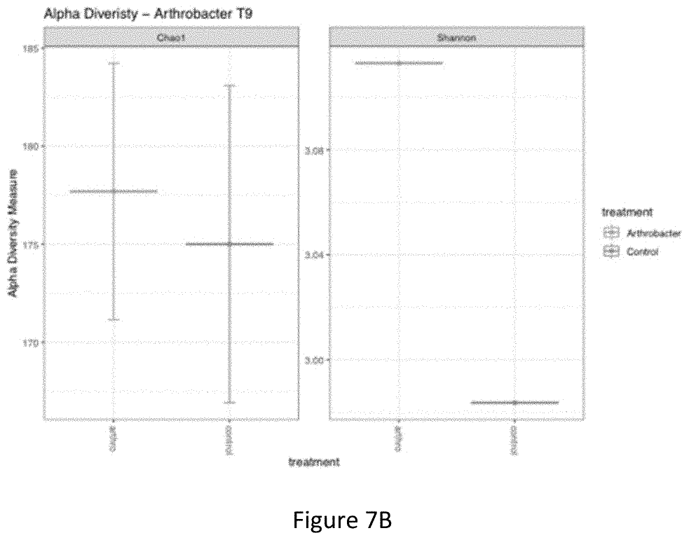

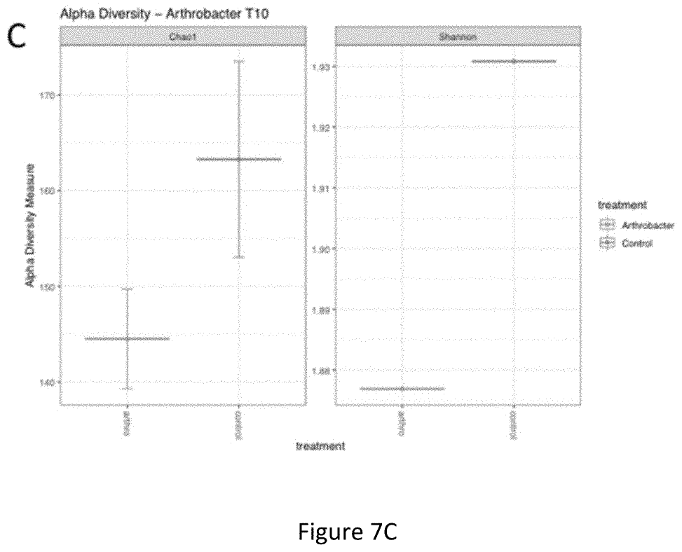

There was no significant difference found in BSFL microbiome species richness at day 7 or 9 as shown in FIGS. 7A and 7B); however, a significant difference in richness was found for day 10 as shown in FIG. 7C. When samples were weighted on abundance, there was a statistically significant difference between treatment and control samples at all timepoints (FIG. 7). At day 7 and 9, there was a higher abundance of species found in Arthrobacter supplemented samples, but this was reversed at day 10, at which time control samples showed an increased abundance in species.

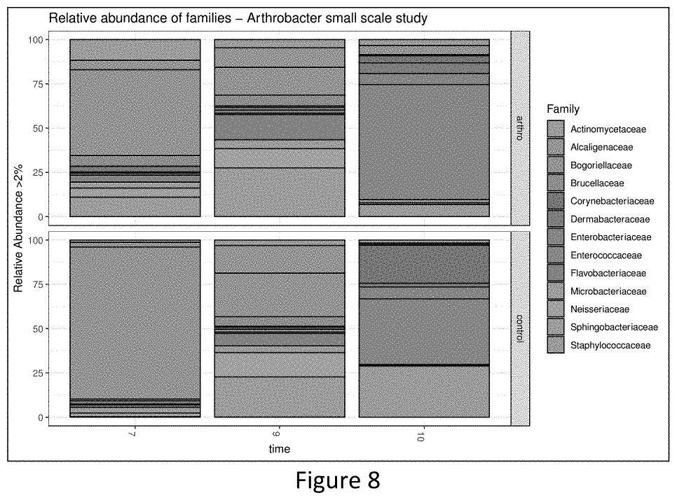

FIG. 8 shows the relative abundance of BSFL associated bacterial families. On day 7, the treatment group showed greater diversity and had increased abundance from every family represented, with the exception that the control group was composed of 39% more Bogoriellaceae and 9% more Enterococcaceae. Of those families that were increased in treatments at day7, all showed over 100% increase except for a 11% increase in Microbacteriaceae and a 23% increase in Staphylococcaceae. At T9, the differences were not as apparent. Treatment larvae saw a 21-36% decrease in Alcaligenaceae, Bogoriellaceae, and Neisseriaceae. Treatment larvae had 50% more Actinomycetaceae, 62% more Corynebacteriaceae, 44% more Dermabacteraceae, 26% more Microbacteriaceae, and over 100% increases in Flavobacteriaceae and Staphylococcaceae as compared to control larval associated microbial families. At day 10 another shift in abundance was identified. Bogoriellaceae, which was decreased from the previous timepoint, was over 300% more abundant in treatment groups. Families that saw a decrease in treatments from controls at day 10 were: Alcaligenaceae(66% decrease), Brucellaceae(21% decrease), Corynebaceriaceae(87% decrease), Enterobacteriaceae(40% decrease), Flavobacteriaceae(74% decrease), Neisseriaceae(70% decrease) and Staphylococcaceae(84% decrease).

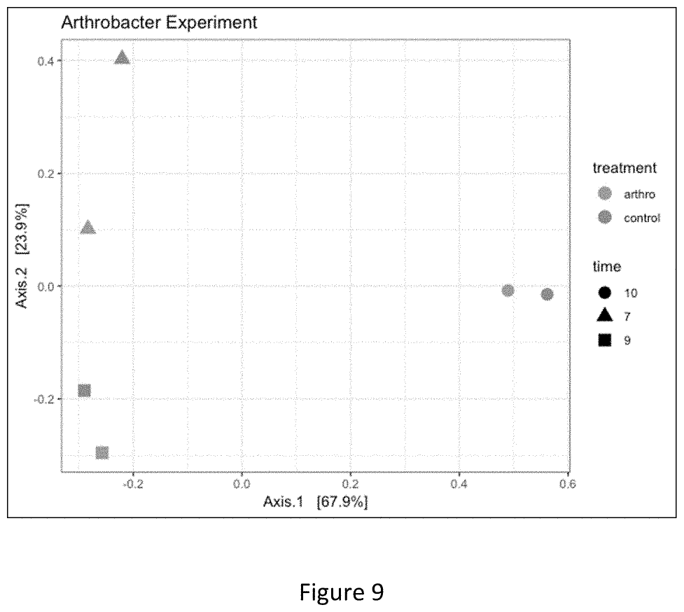

PCoA of β-diversity comparison using Bray Curtis distances revealed significant separation of microbial communities based on time point (p=0.03), Control and treatment samples were similar at days 9 and 10, but the PCoA shows a notable separation at day 7 as shown in FIG. 9.

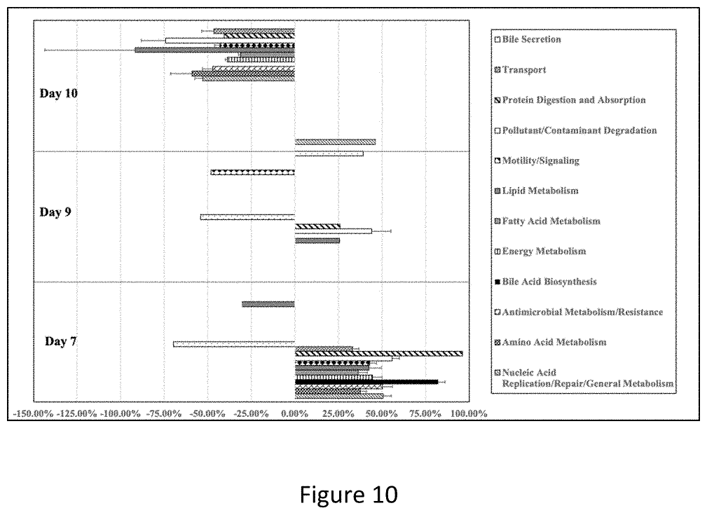

We used PICRUSt to explore relationships between predicted functional gene annotations and identified metagenomes from Arthrobacter supplemented samples compared to controls. From this, 263 annotated genes were identified, compressed into 12 general gene families and used to compare gene abundance between treatment and control groups. Only families with gene abundance within Arthrobacter group at or above 25% change from control are shown in FIG. 10. Percent difference in Arthrobacter group gene abundance compared to control samples at 7 days revealed predicted genes enriched for functions involved in protein digestion and absorption (96.05%), Bile acid biosynthesis (82.21%), pollutant/contaminant digestion (55.82%), nucleic acid repair/replication/general metabolism (50.61%), antimicrobial metabolism/resistance (50.26%), motility and signaling (42.73%), some genes involved in lipid metabolism (42.39%), energy metabolism (44.47%), fatty acid metabolism (36.17%), amino acid metabolism (37.59%), and membrane transport (33.00%). However, compared to controls at 7 days, samples showed a decrease in functions for bile secretion (−69.69%) as well as decrease in some genes for lipid metabolism (−30.21%).

At 9 days, fewer differences in gene abundance were found in the Arthrobacter group compared to control. Genes increased, as compared to control, included those for pollutant/contaminant degradation (43.98%), protein digestion and absorption (25.84%), and lipid metabolism (25.51%), whereas genes for functions involved in bile secretion (−54.21%) and motility and signaling (−48.15%) were decreased compared to control. At day 10, only two functional categories from the Arthrobacter supplemented group were increased compared to control. Those included genes involved in functions for nucleic acid repair/replication/general metabolism (−46.18%) and for bile secretion (39.05%). However, many functional categories within the Arthrobacter supplementation group showed a decrease in percent abundance compared to control (FIG. 6). These included genes involved in lipid metabolism (−91.59%), pollutant/contaminant degradation (−74.29%), amino acid metabolism (−59.07%), nucleic acid repair/replication/general metabolism (−52.97%), antimicrobial metabolism and resistance (−47.17%), membrane transport (−46.63%), motility and signaling (−43.15%), protein digestion and absorption (−40.59%), energy metabolism (−38.82%), and fatty acid metabolism (−31.09%).

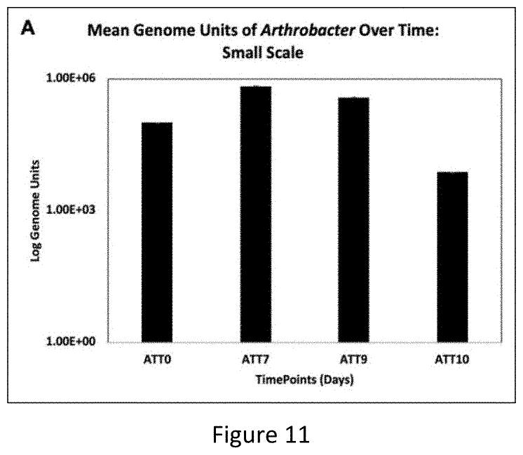

Arthrobacter was detected by qPCR in the treatment group larval guts over time as shown in FIG. 11, where there was a slight increase from the initial inoculum within the first 7 days (from 1×10⁵ CFU to 6.85×10⁵ CFU). Arthrobacter was also detected in larval guts on day 9 (3.78×10⁵ CFU) and on day 10, though had decreased by two logs on day 10 of the experiment (7.36×10³).

### Example 3

Bifidobacterium breve Supplementation Experiment: Small Scale

A similar treatment plan was followed for Bifidobacterium breve as was described above for Example 2, the small scale Arthrobacter supplementation experiment, except instead of 300 larvae per cup only 100 larvae were placed into each treatment container. An identical feeding plan and percent bacteria were used. Instead of placing the bacterial supplement into each day's diet and then feeding, Bifidobacterium breve was grown on plates anaerobically until 1% by weight of the total diet could be replaced with the supplement. The entire volume of diet required for a 10-day experiment was weighed out and prepared with the appropriate volume of water in advance. The diet was placed in an anaerobic chamber, maintained by anaerobic packs that were changed out daily. The inoculum amount was added into the diet and allowed to colonize and the diet required for the entire experiment was kept at growing temperature. The BSFL larvae in their treatment containers were left on the benchtop at room temperature with a perforated plastic wrap on top of each cup to prevent escape. In order to make sure temperature was not significantly different between the control and treatment groups, the diet and water for the control groups was also kept in the same incubator and was the same temperature during feedings.

### Results

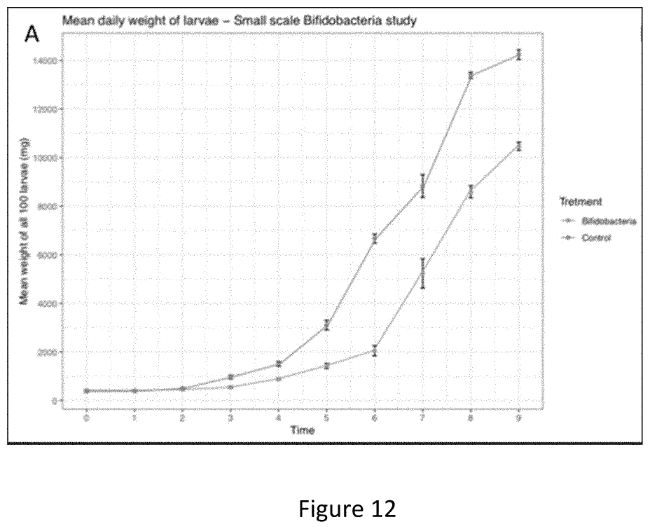

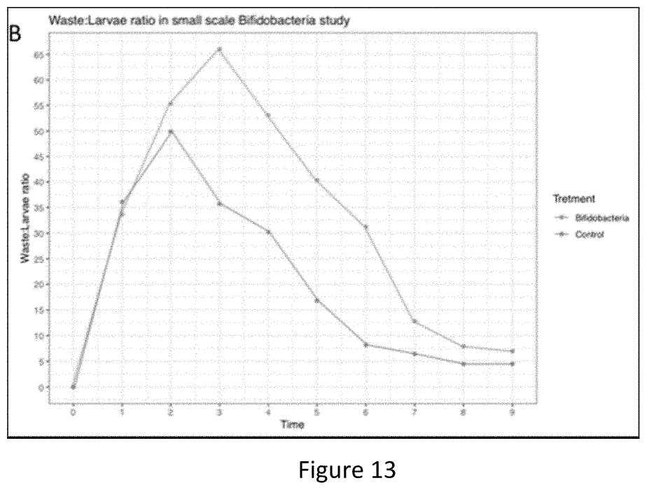

Supplementing with Bifidobacterium yielded lower weights over time compared to control BSFL (A) as shown in FIG. 12. Additionally, supplemented larvae appeared weak, slow, and discolored (data not shown). Also, the treatment BSFL waste:larvae ratio was much lower than control across all timepoints as shown in FIG. 13.

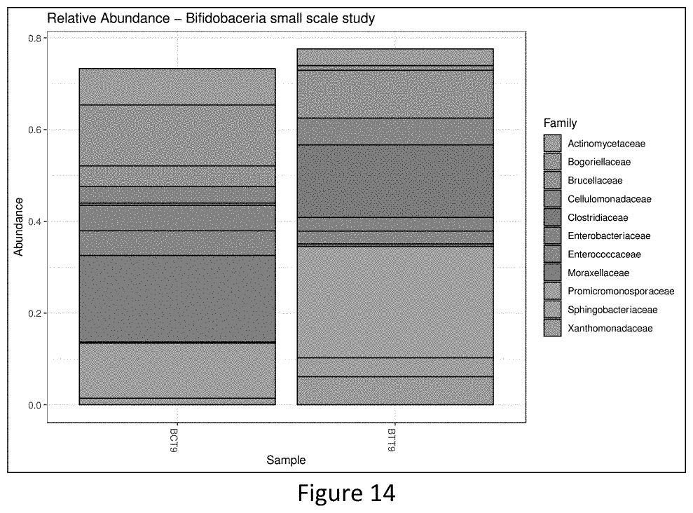

As shown in FIG. 14, relative abundance of control groups showed an increased amount of Actinomycetaceae (97% increase), Bogoriellaceae (99% increase), Brucellaceae (85% increase), Cellulomonadaceae (89% increase) Enterobacteriaceae (96% increase), Enterococcaceae (96% increase), Moraxellaceae (99% increase), Sphingobacteriaceae (98% increase), and Xanthomonadaceae (72% increase) compared to treatment groups. Treatment B. breve supplemented BSFL showed an increase in Clostridiaceae (107.9% increase) and Promicromonosporaceae (510% increase) compared to controls at day 9.

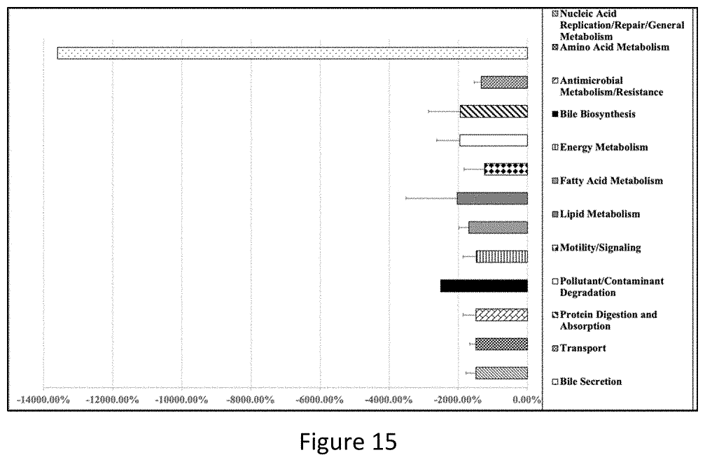

PICRUSt identified 168 genes that were above 25% change from controls on day 9 of the experiment. These were compressed into 12 general gene families, as shown in FIG. 15. Samples with B. breve supplementation showed decrease in predicted functions for all gene families, compared to control. These included percent decrease in bile secretion (−13,601%), transport (−1,333%), protein digestion and absorption (−1,946%), pollutant/contaminant degradation (−1,958%), motility/signaling (−1,231%), lipid metabolism (−2,023%), fatty acid metabolism (−1,696%), energy metabolism (−1,491%), bile biosynthesis (−2,503%), antimicrobial metabolism/resistance (−1,491%), amino acid metabolism (−1,482%), and nucleic acid replication and repair, and general metabolism (1,488%).

### Example 4

Arthrobacter AK19 and R. rhodochrous 21198 Supplementation: Industrial Scale

The industrial scale experiments were conducted using Arthrobacter AK19 and R. rhodochrous 21198. Gainesville diet was used as a diet base where either 8.0 g of R. rhodochrous 21198 or Arthrobacter AK19 was added to 6 kg of diet per pan (four pans per treatment or control, N=16 total), stirring with gloves for 30 seconds to disperse the supplement. Following this, approximately 10,000 larvae were added to each pan of either non-supplemented or supplemented. Each treatment condition (Arthrobacter-supplemented, Rhodococcus-supplemented, and control) had 4 replicates. The larvae were allowed to feed constantly on the initially placed food source. The pans were mixed daily to ensure that temperature spikes due to composting action did not occur. Every three days, a subset of 500 larvae was removed from the pans, weighed, and frozen for later analysis. A sample of the waste was also collected and frozen for later analysis. The experiment was carried out for 10 days. On the final day, 500 larvae from each replicate were removed, weighed, and frozen. The rest of the larvae were sifted from the waste and the total mass of the larvae, as well as the total mass of waste, was weighed. One liter of waste was saved and dried down to determine moisture content.

### Results

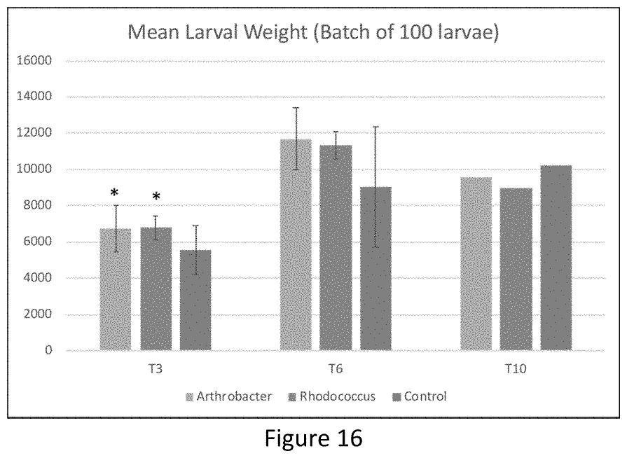

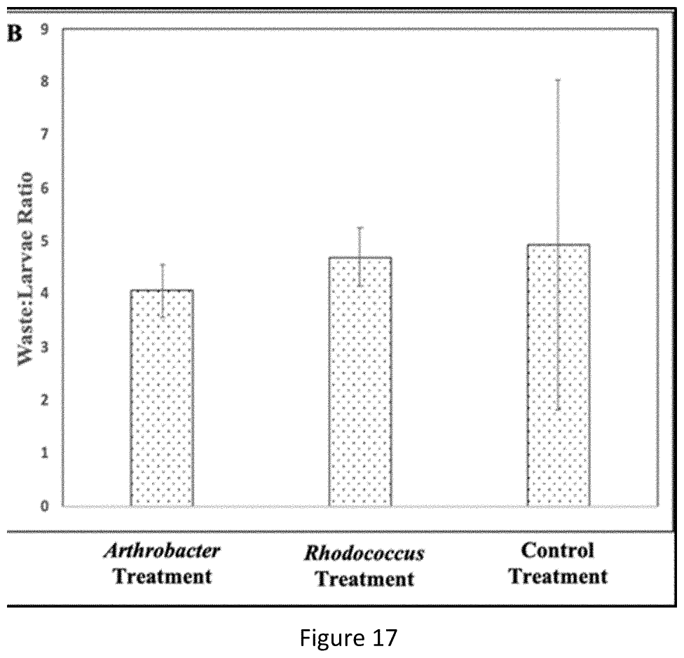

The mean weights of 100 larvae from each treatment are shown in FIG. 16. Arthrobacter and Rhodococcus supplemented larvae were not statistically different from each other for the duration of the study. However, both treatment groups weighed statistically significantly more than control larvae at day 3 (p=0.02) where Arthrobacter treated larvae were 21.6% larger than controls, and Rhodococcus treated larvae were 22.2% larger than controls. At day 6, treatment groups were not statistically different from controls, likely due to large variation in control larvae (p=0.06), though Arthrobacter treated larvae were 29% larger than controls, and Rhodococcus treated larvae were 25% larger than controls. At day 10, control larvae weighed 6.3% more than Arthrobacter treatments, and 12.0% more than Rhodococcus treated larvae. The waste:larvae ratio from day 10 was not significantly different between treatments (p=0.793) shown in FIG. 17.

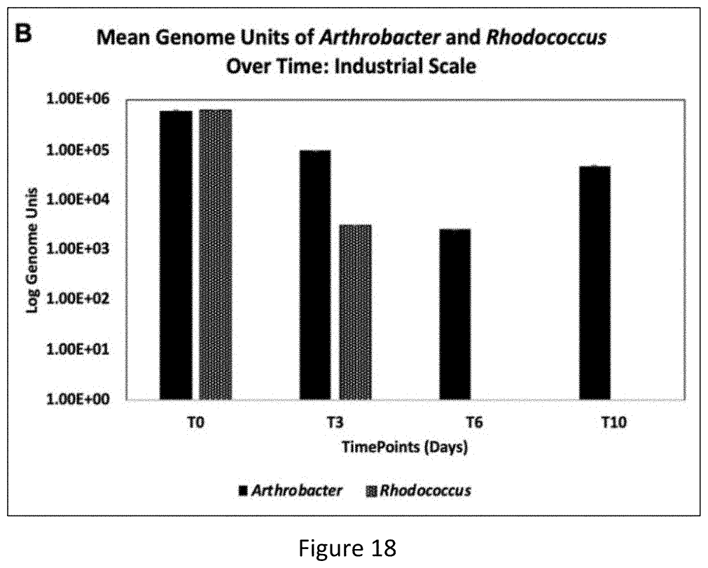

Arthrobacter and Rhodococcus were detected by qPCR through time to determine growth of the supplemented bacteria within the larval guts. The results are shown in FIG. 18. We found an initial decrease of one log (from 6.0×10⁵ CFU initial inoculum to 9.76×10⁴ CFU on day 3) from the initial Arthrobacter inoculum within the first three days of the experiment. There was continued decrease by another log by day 6 of the experiment (2.56×10³ CFU/. However, 4.72 ×10⁴ Arthrobacter genome units were detected at day 10.

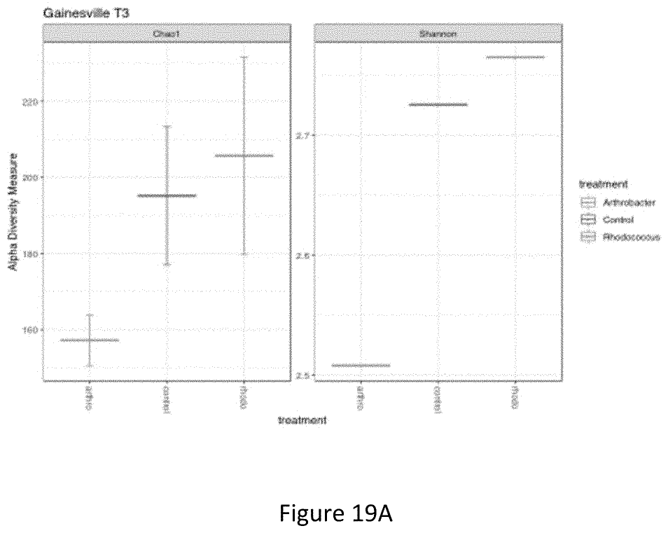

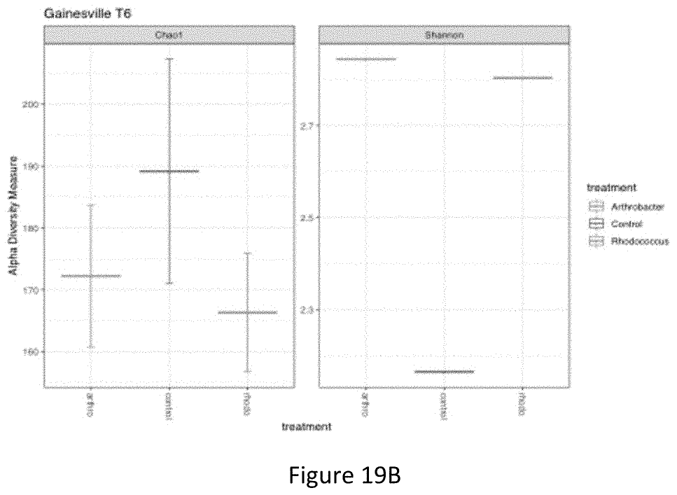

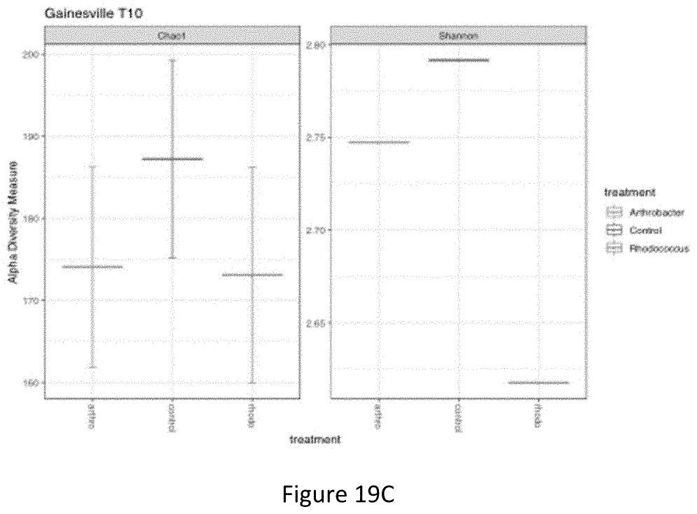

As shown in FIG. 19, richness was lower for larval gut microbiomes from the Arthrobacter treatment on day 3 than for larval gut microbiomes from the control or Rhodococcus treatments but was similar to Rhodococcus on days 6 and 10 where control richness was higher than both bacterial treatments. The Arthrobacter treatment also showed lower evenness than the control and Rhodococcus treatments on day 3. This shifted on days 6 and 10 where both Arthrobacter and Rhodococcus treatments showed higher evenness than control. There was no statistical significance with any of the alpha diversity metrics (p=0.4).

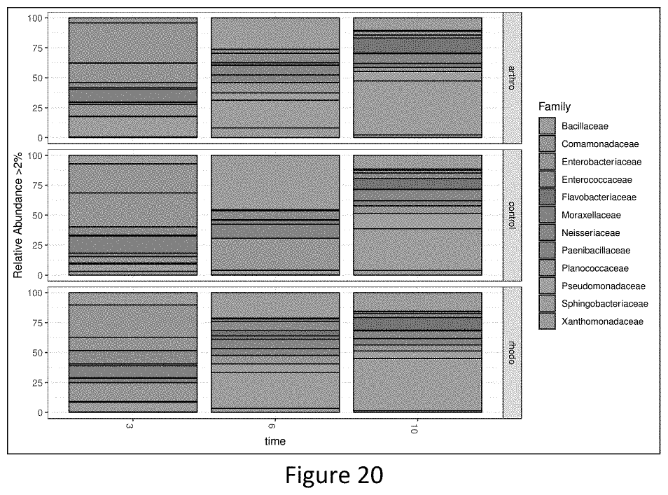

At day 3, larvae fed with Arthrobacter and Rhodococcus had similar relative abundance compared to control as shown in FIG. 20. At day 6, compared to control, Arthrobacter treated larvae had 894% more Enterobacteriaceae, 12% more Enterococcaceae, 766% more Flavobacteriaceae, 3% more Moraxellaceae, 157% more Neisseriaceae, 2295% more Pseudomonadaceae, 564% more Sphingobacteriaceae, 4794% more Xanthomonadaceae, 38% less Bacillaceae, 27% less Comamonadaceae, 41% less Paenibacillaceae, and 65% less Planococcaceae. Day 6 Rhodococcus treated larvae, when compared to controls had 97% more Pseudomonadaceae, 89% more Sphingobacteriaceae, 95% more Flavobacteriaceae, 24% more Enterbacteriaceae, 2% more Paenibacillaceae, 10% more Planococcaceae, 95% more Xanthomonadaceae, 45% Bacillaceae, 87% less Enterobacteriaceae, 93% less Moraxellaceae, and 5% less Neisseriaceae. At day 10, species richness was similar in all groups. However, differences in relative abundance were noted from day 6 to day 10. For instance, Arthrobacter had notable differences at day 10 with 52% less abundance in Bacilliaceae, 72% less Planococcaceae, 72% less Entercoccaceae, 60% less Paenibacillaceae, 60% less Comamonadaceae, 34% less Enterbacteriaceae, but 35% more Moraxellaceae in controls than in Arthrobacter supplemented larvae.

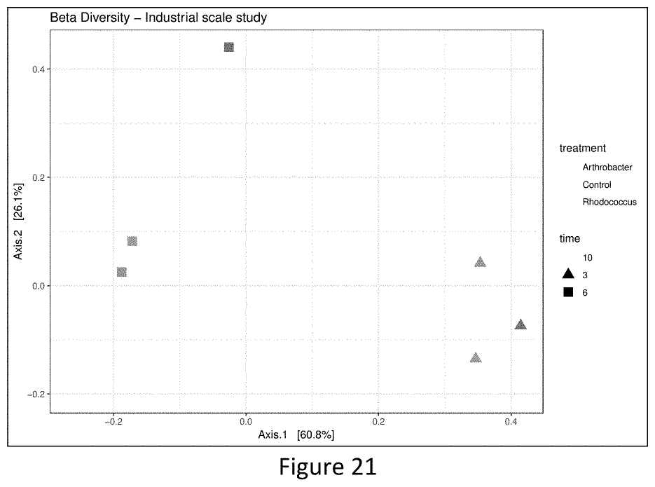

Permutational analysis of variance (ANOVA) of Bray-Curtis beta diversities indicated that timepoint differences explained microbial taxonomic variation (permutation test, p=0.004, 99,999 permutations), and by treatment where each sample showed statistical variance from the other (p<0.001, 99,999 permutations). PCoA of (3-diversity comparison using Bray Curtis distances revealed significant separation of microbial communities based on timepoint (p=0.03). Control and treatment samples were similar at days 3 and 10, but the PCoA showed a notable separation at day 6 between the two treatments from the control samples. As shown in FIG. 21.

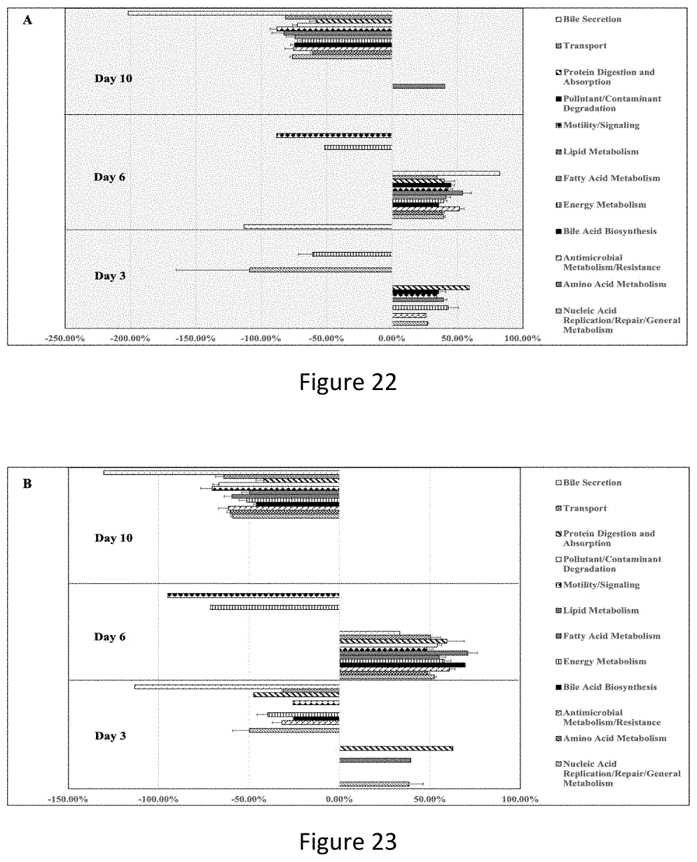

Arthrobacter supplemented B SF larvae microbiomes showed increased percent difference in predicted genes compared to control samples for functions involved in protein digestion and absorption (58.93%), energy metabolism (42.77%), lipid metabolism (39.28%), pollutant/contaminant digestion (35.62%), motility and signaling (34.22%), nucleic acid replication/repair/general metabolism (27.19%), and antimicrobial metabolism/resistance (25.97%). Additionally, other genes for energy metabolism (−60.87%), nucleic acid replication/repair/general metabolism (−108.80%), and bile secretion (−113.19) were decreased compared to control (FIG. 13). At day 6, Arthrobacter treatments showed enrichment in all general gene families, with the highest percent change from control being bile secretion (82.26%), followed by lipid metabolism (53.91%), antimicrobial metabolism and resistance (51.47%), pollutant/contaminant degradation (45.01%), motility and signaling (42.50%), fatty acid metabolism (41.29%), protein digestion and absorption (39.83%), energy metabolism (39.33%), amino acid metabolism (38.12%), bile acid synthesis (35.73%), and transport (34.42%). Only two gene families were decreased from control at day 6 including motility and signaling (−88.48%) and energy metabolism (−51.53%, FIG. 13). At day 10, Arthrobacter treatments only had increases in genes functionally predicted for lipid metabolism (40.41%). However, genes associated with all gene families were decreased compared to control. Those included genes for bile secretion (−201.94%), motility and signaling (−88.06%), lipid metabolism (−82.63%), transport (−81.46%), nucleic acid replication/repair/general metabolism (−76.07%), antimicrobial metabolism and resistance (−75.27%), bile acid biosynthesis (−74.67%), fatty acid metabolism (−74.36%), pollutant/contaminant degradation (−72.51%), energy metabolism (−68.67%), amino acid metabolism (−60.46%), and protein digestion and absorption (−57.99%). This data is show in FIG. 22.

At day 3, Rhodococcus treated BSF larvae microbiomes showed percent increase compared to control in genes involved in protein digestion and absorption (62.69%), nucleic acid replication/repair/general metabolism (38.38%), and lipid metabolism (39.33%). Nine of the 12 gene families were decreased from control including bile secretion (−113.19%), nucleic acid replication/repair/general metabolism (−49.81%), protein digestion and absorption (−47.71%), energy metabolism (−39.69%), antimicrobial metabolism and resistance (−31.75%), transport (−31.15%), pollutant/contaminant degradation (−29.49%), motility and signaling (−25.82%), and bile acid biosynthesis (−25.60%). All gene families were increased compared to control samples, with lipid metabolism being the most increased (71.03%). Following this, bile acid biosynthesis (69.28%), antimicrobial metabolism/resistance (60.74%), protein digestion and absorption (59.38%), energy metabolism (57.88%), fatty acid metabolism (54.96%), pollutant/contaminant degradation (54.12%), nucleic acid replication/repair/general metabolism (52.21%), transport (50.32%), amino acid metabolism (48.63%), motility and signaling (48.20%), and bile secretion (33.29%). Two gene families showed percent decrease compared to control at day 6, including motility and signaling (−95%) and energy metabolism (−71.46%). On day 10, Rhodococcus samples showed no increased genes compared to control. Functional genes for bile secretion (−130.46%), motility and signaling (−70.40%), pollutant/contaminant degradation (−66.75%), transport (−64.09%), antimicrobial metabolism and resistance (−61.48%), amino acid metabolism (−60.46%), fatty acid metabolism (−59.57%), nucleic acid replication/repair/general metabolism (−59.06%), energy metabolism (−51.33%), lipid metabolism (−49.69%), bile acid biosynthesis (−45.87%), and protein digestion and absorption (−41.80%). This data is shown in FIG. 23.

All parameters presented herein including, but not limited to, sizes, dimensions, times, temperatures, pressures, amounts, distances, quantities, ratios, weights, volumes, percentages, and/or similar features and data and the like, for example, represent approximate values and can vary with the possible embodiments described and those not necessarily described but encompassed by the invention. Further, references to ‘a’ or ‘an’ concerning any particular item, component, material, or product is defined as at least one and could be more than one.

The above detailed description and the information above are presented to enable any person skilled in the art to make and use the invention. Specific details have been revealed to provide a comprehensive understanding of the present invention and are used for explanation of the information provided. These specific details, however, are not required to practice the invention, as is apparent to one skilled in the art. Descriptions of specific applications, analyses, materials, components, dimensions, and calculations are meant to serve only as representative examples. Various modifications to the preferred embodiments may be readily apparent to one skilled in the art, and the general principles defined herein may be applicable to other embodiments and applications while still remaining within the scope of the invention. There is no intention for the present invention to be limited to the embodiments shown and the invention is to be accorded the widest possible scope consistent with the principles and features disclosed herein.

While various embodiments of the present invention have been described above, it should be understood that they have been presented by way of example and not limitation. It will be apparent to persons skilled in the relevant art that various changes in form and detail can be made therein without departing from the spirit and scope of the present invention. In fact, after reading the above description, it will be apparent to one skilled in the relevant art how to implement the invention in alternative embodiments. The applicants have described the preferred embodiments of the invention, but it should be understood that the broadest scope of the invention includes such modifications as additional or different methods and materials. Many other advantages of the invention will be apparent to those skilled in the art from the above descriptions and the subsequent claims. Thus, the present invention should not be limited by any of the above-described exemplary embodiments.

The processes, devices, products, apparatus and designs, systems, configurations, methods and/or compositions of the present invention are often best practiced by empirically determining the appropriate values of the operating parameters or by conducting simulations to arrive at best design for a given application. Accordingly, all suitable modifications, combinations, and equivalents should be considered as falling within the spirit and scope of the invention.

1. A method of feeding an animal comprising a step of feeding the animal a feed comprising oleaginous microbes selected from species of Arthrobacter, species of Rhodococcus and combinations thereof.

2. The method of claim 1, wherein the oleaginous microbes comprise from about 0.05 wt. % to about 20 wt. % of a total weight of the feed.

3. The method of claim 1, wherein the oleaginous microbes comprise one or more bacteria selected from Arthrobacter AK19 and Rhodococcus rhodochrous 21198.

4. A black soldier fly larva feed comprising:
a bulk feed suitable for a black soldier fly larva; and
a supplement comprising oleaginous microbes; and
wherein the supplement comprises from about 0.05 wt. % to about 20 wt. % of a total weight of the black soldier fly larva feed.

5. The feed of claim 4, wherein the oleaginous microbes are selected from species of Arthrobacter, species of Rhodococcus and combinations thereof.

6. The feed of claim 5, wherein the supplement consists of oleaginous microbes.

7. The feed of claim 4, wherein the oleaginous microbes consist of species of Arthrobacter.

8. The feed of claim 4, wherein the oleaginous microbes consist of species of Rhodococcus.

9. The feed of claim 4, wherein the oleaginous microbes comprise one or more bacteria selected from Arthrobacter AK19 and Rhodococcus rhodochrous 21198.

10. The feed of claim 4, wherein the bulk feed comprises one or more of alfalfa meal, corn meal, wheat bran, brewer's grain, manure, or food waste.

11. The feed of claim 10, wherein the bulk feed is about 30% alfalfa meal, about 20% corn meal, and about 50% wheat bran.

12. The feed of claim 4, wherein the supplement comprises from about 0.1 wt. % to about 10 wt. % of the total weight of the black soldier fly larva feed.

13. The feed of claim 4, wherein desiccated oleaginous microbes are rehydrated for use in the supplement.

14. A method of increasing the growth, development, or nutritional value of black soldier fly larva, comprising steps of:
mixing a bulk feed suitable for black soldier fly larvae with a supplement comprising oleaginous microbes to create a black soldier fly larva feed wherein the supplement is from about 0.05 wt. % to about 20 wt. % of a total weight of the black soldier fly larva feed, and feeding the black soldier fly larva feed to black soldier fly larvae.

15. The method of claim 14, wherein the oleaginous microbes comprise species selected from species of Arthrobacter, species of Rhodococcus and combinations thereof.

16. The method of claim 14, wherein the supplement consists of oleaginous microbes.

17. The method of claim 14, wherein the supplement comprises from about 0.1 wt. % to about 10 wt. % of the total weight of the black soldier fly larva feed.

18. The method of claim 14, wherein the oleaginous microbes comprise one or more bacteria selected from Arthrobacter AK19 and Rhodococcus rhodochrous 21198.

19. The method of claim 13, wherein the feeding occurs starting on day 11 after the black soldier fly larvae hatch.

20. A method of increasing livestock production comprising a step of:
feeding livestock black soldier fly larva that were fed with the black soldier fly larva feed of claim 4.
